(19) United States
(12) Patent Application Publication
(10) Pub. No.: US 2022/0043005 A1
(43) Pub. Date: Feb. 10, 2022

(54) SELENOPROTEIN P IN HEART FAILURE

(71) Applicant: SPHINGOTEC GMBH, Hennigsdorf (DE)

(72) Inventors: Andreas BERGMANN, Berlin (DE); Olle MELANDER, Limhamm (SE); Martin MAGNUSSON, Bjärred (SE)

(73) Assignee: SPHINGOTEC GMBH, Hennigsdorf (DE)

(21) Appl. No.: 17/415,242

(22) PCT Filed: Dec. 20, 2019
(86) PCT No.: PCT/EP2019/086844

§ 371 Date: Jun. 17, 2021

(30) Foreign Application Priority Data
Dec. 20, 2018 (EP) 18214780.1

(51) Int. Cl. G01N 33/68; A61K 33/04; A61P 9/04

(52) U.S. Cl. CPC G01N 33/6893; A61K 33/04; G01N 2800/325; G01N 2800/50; A61P 9/04

(57) ABSTRACT

A method for assessing risk in a subject having heart failure that is (i) risk for getting a cardiovascular event and/or (ii) risk of worsening heart failure condition and/or (iii) assessing risk for mortality, and/or (iv) assessing risk of hospitalization or re-hospitalization due to heart failure, involves:
a) determining the level and/or amount of Selenoprotein P and/or fragments thereof in a sample from the subject,
b) correlating the determined level and/or the amount of Selenoprotein P and/or fragments thereof in a subject having heart failure with one or more of the risks (i) to (iv) mentioned above.

Subject matter of the present invention includes stratification of patients and treatment methods for heart failure patients at high risk (i) for getting a cardiovascular event and/or (ii) of worsening heart failure condition and/or (iii) for mortality, in particular cardiovascular mortality, and/or (iv) of hospitalisation or re-hospitalisation due to heart failure.

Subject matter of the present invention is a method for assessing a risk in a subject having heart failure that is (i) the risk of getting a cardiovascular event and/or (ii) the risk of worsening heart failure condition and/or (iii) the risk for mortality, in particular cardiovascular mortality, and/or (iv) the risk of hospitalisation or re-hospitalisation due to heart failure, comprising
a) determining the level and/or the amount of Selenoprotein P and/or fragments thereof in a sample of said subject,
b) correlating the determined level and/or the amount of Selenoprotein P and/or fragments thereof in a subject having heart failure with (i) the risk for getting a cardiovascular event and/or (ii) with the risk of worsening heart failure condition and/or (iii) with the risk for mortality, in particular cardiovascular mortality, and/or (iv) with the risk of hospitalisation or re-hospitalisation due to heart failure.

Subject matter of the present invention includes stratification of patients and treatment methods for patients having heart failure at high risk (i) for getting a cardiovascular event and/or (ii) of worsening heart failure condition and/or (iii) for mortality, in particular cardiovascular mortality and/or (iv) of hospitalisation or re-hospitalisation due to heart failure.

### STATE OF THE ART

Selenoprotein P (abbreviations Sepp1, SeP, SELP, SePP) is a plasma selenoprotein, that serves as selenium nutritional marker and its plasma level falls fall as the severity of selenium deficiency increases (Yang, Hill et al. 1989, Renko, Werner et al. 2008).

Because selenium functions through selenoproteins, it has been proposed that optimum health would be achieved if enough of the trace element was supplied to prevent selenium from becoming the limiting factor in selenoprotein synthesis. Determination of selenoprotein optimization has become the major technique used to assess the selenium nutritional requirement (Burk and Hill 2009).

So far, over 25 selenoproteins have been identified that play diverse roles in the regulation of cellular redox processes (Liu, Xu et al. 2017). They are expressed in a variety of tissues and cells and exhibit numerous functions, e.g. glutathione peroxidases (GPx) detoxify intracellular hydrogen peroxide thus protecting the cell from lipoprotein and/or DNA damage while thioredoxin reductases (TrxR) regenerate thioredoxin and thereby balance the redox status of the cell (Reeves and Hoffmann 2009).

Selenium plays an essential part in the selenoprotein-induced defense system. Consequently, selenium blood levels have been widely utilized as a biomarker for oxidative stress-associated diseases. Various observational studies have investigated the significance of serum selenium levels on the development of cardiovascular diseases with conflicting results. A dietary supplementation trial with selenium in healthy elderly subjects showed that the cardiovascular mortality was significantly reduced and the cardiac function significantly improved (Alehagen, Johansson et al. 2013) and this was still observed during a follow-up time of 10 years after intervention (Alehagen, Aaseth et al. 2015). Moreover, low selenium levels were associated with future cardiovascular death in patients with acute coronary syndrome but not in patients with stable angina pectoris (Lubos, Sinning et al. 2010). In contrast, meta-analyses of several selenium supplementation trials reported that there were no statistically significant effects of selenium supplementation on cardiovascular mortality and all fatal and non-fatal cardiovascular disease events (Flores-Mateo, Navas-Acien et al. 2006, Rees, Hartley et al. 2013). In summary, the results from randomized trials to date have been inconsistent and the role of selenium supplementation in cardiovascular disease prevention is inconclusive.

The difference in the baseline selenium status of the populations studied and the dose of selenium supplementation might partially account for the lack of consistency in trial studies. Selenium supplementation may benefit people with low baseline selenium status, but have no effect or even an adverse effect on the cardiovascular system in people with adequate-to-high status. For example, supplementation of additional selenium in people who already have adequate selenium intake might increase their risk of type-2 diabetes (Rayman and Stranges 2013). Thus, a U-shaped association between selenium status and cardiovascular disease risk may be reasonable (Bleys, Navas-Acien et al. 2008).

Selenium deficiency can induce heart failure (Saliba, El Fakih et al. 2010). Heart failure was associated with lower selenium level (Kosar, Sahin et al. 2006) and serum selenium was reduced in African-American patients with congestive heart failure (Arroyo, Laguardia et al. 2006). In contrast, Ghaemian et al. showed that serum selenium levels in congestive heart failure patients were similar to those of controls and the selenium levels did not correlate with the degree of left ventricular dysfunction (Ghaemian, Salehifar et al. 2012). According to a meta analysis, selenium supplementation in general population and high cardiovascular disease risk population did not change all-cause mortality, cardiovascular disease mortality or cardiovascular events (Rees, Hartley et al. 2013).

Selenium supplementation studies (Xia, Hill et al. 2005, Burk, Norsworthy et al. 2006, Meplan, Crosley et al. 2007) indicate that Selenoprotein P plasma level is the best easily accessible marker of human selenium nutritional status. A highly significant correlation was found between serum selenium and Selenoprotein-P levels (Andoh, Hirashima et al. 2005). However, once the nutritional requirement is met, Selenoprotein P level concentration does not reflect additional increases in selenium intake.

Selenoprotein P is a secreted glycoprotein that contains most of the selenium in plasma (Read, Bellew et al. 1990, Hill, Xia et al. 1996). With respect to its selenium content, Selenoprotein P can be divided into two domains. The N-terminal domain, approximately two-thirds of the amino acid sequence, contains 1 selenocysteine (U) in a U-x-x-C redox motif. The shorter C-terminal domain contains multiple selenocysteines, e.g. nine in rats, mice, and humans.

Full-length Selenoprotein P is present in plasma but so are shortened forms that have reduced selenium content. Selenoprotein P purified from rat plasma is present as four isoforms. In addition to the full-length isoform that contains ten selenocysteine residues, shorter isoforms are present that terminate at the second, third, and seventh selenocysteine positions. These isoforms contain one, two, and six selenocysteine residues, respectively (Himeno, Chittum et al. 1996, Ma, Hill et al. 2002). There is evidence for the existence of Selenoprotein P isoforms in the mouse (Hill, Zhou et al. 2007) and the human (Akesson, Bellew et al. 1994), respectively. Structurally, human Selenoprotein P is a protein containing 381 amino acid residues (SEQ ID NO. 1) of which ten are predicted to be selenocysteine residues at positions 59, 300, 318, 330, 345, 352, 367, 369, 376 and 378.

Its secreted form (after cleavage of the signal sequence) consists of 362 amino acid residues (SEQ ID NO. 2) and may contain post-translational modifications, which can include phosphorylation and multiple sites of glycosylation. Moreover, several fragments including fragments containing the N- or C-terminal part of Selenoprotein P have been identified (Hirashima, Naruse et al. 2003, Ballihaut, Kilpatrick et al. 2012).

The liver produces most of the Selenoprotein P in plasma, where its turnover is rapid.

Selenoprotein P is also expressed in other tissues and is presumably secreted by them (Hill, Lloyd et al. 1993, Yang, Hill et al. 2000). The liver acquires selenium from several sources and apportions it between selenoprotein synthesis and excretion from the organism.

Specifically, liver synthesizes its intrinsic selenoproteins as well as the secreted selenium molecules Selenoprotein P and excretory metabolites. Whole-body selenium, thus, appears to be regulated in the liver by the distribution of metabolically available selenium between the pathways of selenoprotein synthesis and selenium excretory metabolite synthesis.

Elevated circulating Selenoprotein P levels have been reported in patients with type 2 Diabetes mellitus and prediabetes and were shown to be related to atherosclerosis (Yang, Hwang et al. 2011). Moreover, Selenoprotein P levels concentrations were increased in overweight and obese patients (Chen, Liu et al. 2017). In contrast, Selenoprotein P level concentration is decreased in sepsis and is presumably the cause of the decline in selenium level (Hollenbach, Morgenthaler et al. 2008) or a decreased release of the trace element by the liver (Renko, Hofmann et al. 2009). Significantly decreased circulating Selenoprotein P levels that were associated to the metabolic syndrome status were also found in patients with documented cardiovascular disease (Gharipour, Sadeghi et al. 2017).

There is very limited data on Selenoprotein P in heart failure. Strauss et al. determined Selenoprotein P levels in patients with cardiovascular disease, including heart failure, and found increased Selenoprotein P levels in patients with heart failure compared to patients without heart failure (Strauss, Tomczak et al. 2018). An association between Selenoprotein P and measures of outcome has not been investigated in these patients.

Several Selenoprotein P quantification methods by antibody-based assays are known: a radioimmunoassay (Hill, Xia et al. 1996), an enzyme-linked immunosorbent assay (Andoh, Hirashima et al. 2005), a very sensitive chemiluminescence immunoassay (Hollenbach, Morgenthaler et al. 2008) and very recently sandwich SELENOP-ELISA that was calibrated against a standard reference material (Hybsier, Schulz et al. 2017).

An increased risk for all cause mortality in patients with mainly diabetes exhibiting decreased plasma Selenoprotein P values has been described in WO 2015/185672.

Moreover, it was shown that the detection of Selenoprotein P can be used to assess the risk in a healthy subject for getting a first cardiovascular event or assessing the risk for cardiovascular mortality (PCT/EP2018/079030).

Subject matter of the present invention is a method for assessing a risk in a subject having heart failure that is (i) the risk for getting a cardiovascular event and/or (ii) the risk of worsening heart failure condition and/or (iii) the risk for mortality, in particular cardiovascular mortality, and/or (iv) the risk of hospitalisation or re-hospitalisation due to heart failure, comprising
a) determining the level and/or the amount of Selenoprotein P and/or fragments thereof in a sample of said subject and
b) correlating the determined level and/or the amount of Selenoprotein P and/or fragments thereof in a sample of a subject having heart failure with (i) the risk for getting a cardiovascular event and/or (ii) with the risk of worsening heart failure condition and/or (iii) with the risk for mortality (preferably the risk of mortality within one year), in particular cardiovascular mortality, in a subject having heart failure and/or (iv) with the risk of hospitalisation or re-hospitalisation (preferably within 30 days) due to heart failure.

The risk in a subject having heart failure for getting a cardiovascular event and/or (ii) the risk of worsening heart failure condition and/or (iii) the risk for mortality, in particular cardiovascular mortality, and/or (iv) the risk of hospitalisation or re-hospitalisation due to heart failure is enhanced respectively if the level and/or the amount of Selenoprotein P and/or fragments thereof in a sample of said subject is decreased. In a particular embodiment said before mentioned risks are enhanced if the level and/or the amount of Selenoprotein P and/or fragments thereof in a sample of said subject is below a respective threshold.

Risk for mortality may mean risk for cardiovascular mortality. Cardiovascular mortality means cardiovascular death related to stroke, myocardial infarction, or acute heart failure.

Worsening of heart failure may result in hospitalisation and is defined as (Clark, Cherif et al. 2018):
(i) clinical worsening, i.e. abrupt occurrence of symptoms and signs of heart failure, such as the development of pulmonary oedema, and need for additional intravenous or mechanical therapy and/or
(ii) gradual deterioration of acute heart failure despite therapy and/or
(iii) failure to respond to standard treatment.

In one embodiment, said cardiovascular event may be selected from the group comprising acute decompensated heart failure, atherosclerosis, hypertension, cardiomyopathy and myocardial infarction and said cardiovascular mortality is selected from cardiovascular death related to myocardial infarction, or acute heart failure.

In one specific embodiment of the invention, said patient has a chronic heart failure, and that cardiovascular event is an acute decompensated heart failure.

In one embodiment, said cardiovascular event may be selected from the group comprising acute decompensated heart failure, atherosclerosis, hypertension, cardiomyopathy and myocardial infarction, but said cardiovascular event is not stroke and said cardiovascular mortality is selected from cardiovascular death related to myocardial infarction, or acute heart failure, but said cardiovascular mortality is not related to stroke.

In a specific embodiment of the invention said cardiovascular event is an acute cardiovascular event selected from the group comprising myocardial infarction, acute decompensated heart failure, stroke, coronary re-vascularization and cardiovascular death related to myocardial infarction, stroke or acute heart failure.

In a specific embodiment of the invention said cardiovascular event is an acute cardiovascular event selected from the group comprising myocardial infarction, acute decompensated heart failure, coronary re-vascularization and cardiovascular death related to myocardial infarction, or acute heart failure.

In a specific embodiment of the invention said cardiovascular event is an acute cardiovascular event selected from the group comprising myocardial infarction, acute heart failure, coronary re-vascularization, but not stroke, and cardiovascular death related to myocardial infarction, or acute heart failure, but not related to stroke.

Risk for getting a cardiovascular event and/or cardiovascular mortality means the risk of getting an event due to cardiovascular reasons or the risk of dying because of cardiovascular reasons within a certain period of time. In a specific embodiment said period of time is within 10 years, or within 8 years, or within 5 years or within 2.5 years, or within 1 year, or within 6 months, or within 3 months, or within 30 days, or within 28 days.

Risk of a cardiovascular event or cardiovascular mortality means the risk of getting an event due to cardiovascular reasons or the risk of dying because of cardiovascular reasons within a certain period of time, but wherein the cardiovascular event or cardiovascular mortality is not stroke or related to stroke. In a specific embodiment said period of time is within 10 years, or within 8 years, or within 5 years or within 2.5 years, or within 1 year, or within 6 months, or within 3 months, or within 30 days, or within 28 days.

Risk of a cardiovascular event or cardiovascular mortality means the risk of getting an event due to cardiovascular reasons or the risk of dying because of cardiovascular reasons within a certain period of time, but wherein the cardiovascular event or cardiovascular mortality is not stroke or related to stroke. In a specific embodiment said period of time is within 10 years, or within 8 years, or within 5 years or within 2.5 years, or within 1 year, or within 6 months, or within 3 months, or within 30 days, or within 28 days.

It has been shown that the detection of Selenoprotein P can be used to assess the risk in a healthy subject for getting a first cardiovascular event or assessing the risk for cardiovascular mortality (PCT/EP2018/079030; Schomburg et al. 2018. JAMA Cardiology, submitted) by using a certain threshold (e.g. the median). The frequency distribution of Selenoprotein P in a healthy population ranges from 0.4 to 20.0 mg/L with a median concentration of 5.5 mg/L (FIG. 5A). Threshold ranges of Selenoprotein P to assess the risk of healthy subjects for getting a first cardiovascular event or cardiovascular mortality are between 4.0 and 5.5 mg/L.

When compared to a healthy population, the Selenoprotein P concentration of the heart failure population (e.g. HARVEST study), is a much lower concentration ranging between 0.8 and 6.9 mg/L and a median of 3.0 mg/L, where the majority of values are well below a threshold for healthy subjects (e.g. 97.3% of heart failure patients are below 5.5 mg/L and 79.7% of heart failure patients are below 4.0 mg/L) (FIG. 5B). Heart failure patients have Selenoprotein P concentrations that are compareable to healthy patients having a risk of getting a cardiovascular event, as those patients have already suffered a cardiovascular event (namely heart failure). Surprisingly, and according to the present invention the low Selenoprotein P concentrations in heart failure patients can further be divided into subgroups, whereas Selenoprotein P concentrations at the lower end of the distribution in heart failure patients have a higher risk of e.g. worsening heart failure or rehospitalization due to heart failure or mortality according to the present invention.

Subject matter of the present invention is a method for assessing a risk in a subject having heart failure that is (i) the risk for getting a cardiovascular event and/or (ii) the risk of worsening heart failure condition and/or (iii) assessing the risk for mortality, in particular cardiovascular mortality, and/or (iv) assessing the risk of hospitalisation or re-hospitalisation due to heart failure, comprising
a) determining the level and/or the amount of Selenoprotein P and/or fragments thereof in a sample of said subject and
b) comparing the determined level and/or the amount of Selenoprotein P and/or fragments thereof in a sample of said subject to a reference level of Selenoprotein P and/or fragments thereof of a reference level.

The term “reference level” is well known in the art. Preferred reference levels can be determined by the skilled person without further ado. Preferably, the term “reference level” herein refers to a predetermined value for the respective biomarker. In this context “level” encompasses the absolute amount, the relative amount or concentration as well as any value or parameter which correlates thereto or can be derived therefrom. Preferably, the reference level is a level which allows for allocating the subject into a group of subjects who are at risk of e.g. getting a cardiovascular event, or into a group of subjects who are not at risk of e.g. getting a cardiovascular event. Thus, the reference level shall allow for differentiating between a subject who is at risk or who is not at risk of e.g. getting a cardiovascular event. As the skilled artisan will appreciate the reference level is predetermined and set to meet routine requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90%, 95% or 98%, respectively. These requirements may also be defined in terms of positive or negative predictive values. Nonetheless, based on the teaching given in the present invention it will always be possible for a skilled artisan to arrive at the reference level meeting those requirements. In one embodiment the reference level is determined in a reference sample or samples from a patient (or group of patients) who are at risk. In another embodiment, the reference level is determined in a reference sample or samples from a patient (or group of patients) who are not at risk of e.g. getting a cardiovascular event. The reference level in one embodiment has been predetermined in reference samples from the disease entity to which the patient belongs. In certain embodiments the reference level can e.g. be set to any percentage between 25% and 75% of the overall distribution of the values in a disease entity investigated.

In other embodiments the reference level can e.g. be set to the median, tertiles or quartiles as determined from the overall distribution of the values in reference samples from a disease entity investigated. In one embodiment the reference level is set to the median value as determined from the overall distribution of the values in a disease entity investigated. The reference level may vary depending on various physiological parameters such as age, gender or subpopulation, as well as on the means used for the determination of Selenoprotein P or fragments thereof referred to herein.

A reference level may be determined by measuring Selenoprotein P or fragments thereof in a reference population. A reference population may be a healthy population, e.g. with no signs and symptoms of heart failure. In a further aspect of the invention, a reference population may be a population of subjects suffering from a disease or disorder, in particular heart failure patients. A reference population may consist of more than one reference subjects. An example of a healthy reference population with respective Selenoprotein P concentrations is given in Schomburg et al. (Schomburg et al. 2018. JAMA Cardiology, submitted).

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of re-hospitalisation due to heart failure as above outlined, wherein (i) the risk for getting a cardiovascular event and/or (ii) the risk of worsening heart failure condition and/or (iii) the risk for mortality and/or (iv) the risk of hospitalisation or re-hospitalisation due to heart failure is enhanced when the determined level and/or the amount of Selenoprotein P and/or fragments thereof in a sample of said subject is below a threshold.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein (i) the risk for getting a cardiovascular event and/or (ii) the risk of worsening heart failure condition and/or (iii) the risk for mortality and/or (iv) the risk of hospitalisation or re-hospitalisation due to heart failure is enhanced when said level and/or the amount of Selenoprotein P and/or fragments thereof in said sample is below a threshold, wherein said threshold is between 2.0 and 4.4 mg/L, preferably between 2.3 and 3.8 mg/L, more preferably between 2.6 and 3.4 mg/L, more preferably between 3.0 and 3.3 mg/L, most preferred said threshold is 3.3 mg/L.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of re-hospitalisation due to heart failure as above outlined, wherein (i) the risk for getting a cardiovascular event and/or (ii) the risk of worsening heart failure condition and/or (iii) the risk for mortality and/or (iv) the risk of re-hospitalisation due to heart failure is enhanced when said level and/or the amount of Selenoprotein P and/or fragments thereof in said sample is below a threshold, wherein said threshold has been determined by the calculation of receiver operating characteristic curves (ROC curves), plotting the true positive rate (sensitivity, “disease” population e.g. subjects who did develop the condition) against the false positive rate (1-specificity, “normal” population e.g. subjects who did not develop the condition) at various threshold value settings.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of re-hospitalisation due to heart failure as above outlined, wherein (i) the risk for getting a cardiovascular event and/or (ii) the risk of worsening heart failure condition and/or (iii) the risk for mortality and/or (iv) the risk of re-hospitalisation due to heart failure is enhanced when said level and/or the amount of Selenoprotein P and/or fragments thereof in said sample is below a threshold, wherein said threshold is below 4.4 mg/L, preferably below 3.8 mg/L even more preferably below 3.4 mg/L, most preferred equal to or below 3.3 mg/L.

Therefore, a threshold range is useful between 2.2 and 4.4 mg/L. These thresholds are related to the calibration method mentioned in the examples.

All thresholds and values have to be seen in relation to the test and the calibration used according to the Examples. A person skilled in the art may know that the absolute value of a threshold might be influenced by the calibration used. This means that all values and thresholds given herein are to be understood in context of the calibration used.

Threshold levels may be determined by measuring samples from subjects who did develop a certain condition (e.g. a cardiovascular event) and samples from subjects who did not develop the condition. One possibility to determine a threshold is the calculation of receiver operating characteristic curves (ROC curves), plotting the true positive rate (sensitivity, “disease” population, e.g. subjects who did develop the condition) against the false positive rate (1-specificity, “normal” population e.g. subjects who did not develop the condition) at various threshold value settings. A distribution of the marker levels for subjects developing or not developing a certain condition will likely overlap. Under such conditions, a test does not absolutely distinguish “normal” from “disease” with 100% accuracy, and the area of overlap indicates where the test cannot distinguish normal from “disease”. A threshold is selected, above which (or below which, depending on how a marker changes with the “disease”) the test is considered to be abnormal and below which the test is considered to be normal. The area under the ROC curve (AUC) is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art (Hanley et al. 1982. Radiology 143: 29-36). Preferably, a threshold is selected to provide an AUC of greater than about 0.5, more preferably greater than about 0.7, still more preferably greater than about 0.8, even more preferably greater than about 0.85, and most preferably greater than about 0.9. The term “about” in this context refers to +/−5% of a given measurement. The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The AUC is a measure of the probability that the measured marker level will allow correct identification of a disease or condition. Thus, the AUC can be used to determine the effectiveness of the test. The odds ratio (OR) is a measure of effect size, describing the strength of association or non-independence between two binary data values (e.g. the ratio of the odds of an event occurring in test negative group to the odds of it occurring in the test positive group).

Threshold levels can be obtained for instance from a Kaplan-Meier analysis, where the occurrence of a disease or the probability of a serious condition and/or death is correlated with the e.g. quartiles of the respective markers in the population. According to this analysis, subjects with marker levels above the 75th percentile have a significantly increased risk for getting the diseases according to the invention. This result is further supported by Cox regression analysis with adjustment for classical risk factors. The highest (or lowest quartile, depending on how a marker changes with the “disease”) versus all other subjects is highly significantly associated with increased risk for getting a disease or the probability of a serious condition and/or death according to the invention.

Other preferred threshold values are for instance the 10th, 5th or 1st percentile of a reference population. By using a higher percentile than the 25th percentile, one reduces the number of false positive subjects identified, but one might miss to identify subjects, who are at moderate, albeit still increased risk. Thus, one might adapt the threshold value depending on whether it is considered more appropriate to identify most of the subjects at risk at the expense of also identifying “false positives”, or whether it is considered more appropriate to identify mainly the subjects at high risk at the expense of missing several subjects at moderate risk.

The person skilled in the art knows how to determine such statistically significant levels.

In one embodiment of the invention the subject is male.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said cardiovascular event is selected from a group comprising myocardial infarction, stroke, coronary re-vascularization and said mortality is cardiovascular mortality.

Subject matter of the present invention is a method for assessing a risk in a patient having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said cardiovascular mortality is selected from cardiovascular death related to myocardial infarction, stroke or acute heart failure.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said level and/or amount of Selenoprotein P and/or fragments thereof has been determined by an immunoassay using at least one binder binding to SEQ ID No. 2.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said at least one binder is an antibody or a fragment thereof.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said level and/or amount of Selenoprotein P and/or fragments thereof has been determined by mass spectroscopy.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said risk for getting a cardiovascular event including death is assessed for a period of time of up to one year.

Subject matter of the present invention is a method for assessing a risk in a subject having heat failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said (ii) risk of worsening heart failure condition and/or (iii) the risk for mortality in said subject is assessed for a period of time of up to one year.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein said risk of hospitalisation or re-hospitalisation due to heart failure is assessed for a period of 30 days.

Subject matter of the present invention is a method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure as above outlined, wherein the sample is a bodily fluid.

A bodily fluid may be selected from the group comprising whole blood, serum, plasma, urine, cerebrospinal liquid (CSF), and saliva.

In a preferred embodiment the sample is a bodily fluid selected from the group comprising whole blood, plasma, and serum.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein the cardiovascular event is not stroke and wherein the cardiovascular mortality is not related to stroke.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein the cardiovascular event is not stroke and wherein the cardiovascular mortality is not related to stroke.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein the determined level and/or the amount of Selenoprotein P and/or fragments thereof is below a threshold and wherein said threshold is between 2.0 and 4.4 mg/L, preferably between 2.3 and 3.8 mg/L, more preferably between 2.6 and 3.4 mg/L, more preferably between 3.0 and 3.3 mg/L, most preferred 3.3 mg/L.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein the determined level and/or the amount of Selenoprotein P and/or fragments thereof is below a threshold and wherein said threshold is between 2.0 and 4.4 mg/L, preferably between 2.3 and 3.8 mg/L, more preferably between 2.6 and 3.4 mg/L, more preferably between 3.0 and 3.3 mg/L, most preferred 3.3 mg/L, wherein the cardiovascular event is not stroke and wherein the cardiovascular mortality is not related to stroke.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein selenium is administered to said subject in a pharmaceutically acceptable amount.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein selenium is administered to said subject in a pharmaceutically acceptable amount, wherein the cardiovascular event is not stroke and wherein the cardiovascular mortality is not related to stroke.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein selenium is administered to said subject in a pharmaceutically acceptable amount and wherein the determined level and/or the amount of Selenoprotein P and/or fragments thereof is below a threshold and wherein said threshold is between 2.0 and 4.4 mg/L, and wherein selenium is administered to said subject in a pharmaceutically acceptable amount to reduce said risks.

Subject matter of the present invention is selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure, wherein such an enhanced risk is determined according to a method according to the present invention as outlined herein, wherein selenium is administered to said subject in a pharmaceutically acceptable amount and wherein the determined level and/or the amount of Selenoprotein P and/or fragments thereof is below a threshold and wherein said threshold is between 2.0 and 4.4 mg/L, and wherein selenium is administered to said subject in a pharmaceutically acceptable amount to reduce said risks, wherein the cardiovascular event is not stroke and wherein the cardiovascular mortality is not related to stroke.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to the invention is performed at least two times.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to the invention is performed at least two times, wherein the cardiovascular event is not stroke and wherein the cardiovascular mortality is not related to stroke.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to the invention is performed as monitoring of said treatment.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to the invention is performed as monitoring of said treatment, wherein the cardiovascular event is not stroke and wherein the cardiovascular mortality is not related to stroke.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to the invention is performed and used for therapy-guidance.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to the invention is performed and used for therapy-guidance, wherein the cardiovascular event is not stroke and wherein the cardiovascular mortality is not related to stroke.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to any of the above embodiments is performed and used as companion diagnostics.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein a method for assessing said risk according to any of the above embodiments is performed and used as companion diagnostics, wherein the cardiovascular event is not stroke and wherein the cardiovascular mortality is not related to stroke.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein the selenium administered is selected from the group comprising selenite, selenate or selenomethionine (L-selenomethionine).

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein the selenium administered is selected from the group comprising selenite, selenate or selenomethionine (L-selenomethionine), wherein the cardiovascular event is not stroke and wherein the cardiovascular mortality is not related to stroke.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein the selenium administered is selected from the group comprising selenite, selenate or selenomethionine (L-selenomethionine) in combination of anti-oxidant co-enzyme Q10 as an essential co-enzyme.

Subject matter of the present invention is a method of treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure according to any of the above-outlined embodiments, wherein the selenium administered is selected from the group comprising selenite, selenate or selenomethionine (L-selenomethionine) in combination of anti-oxidant co-enzyme Q10 as an essential co-enzyme, wherein the cardiovascular event is not stroke and wherein the cardiovascular mortality is not related to stroke.

In said method of treatments the above-mentioned methods for assessing said risks are used including threshold ranges as above mentioned. One threshold may be selenium levels equal or <100 μg/L.

The term “subject” as used herein refers to a living human or non-human organism. Preferably herein the subject is a human subject. The subject is suffering from heart failure.

The term “decreased level” means a level below a certain threshold level. The term “increased level” means a level above a certain threshold level.

The term “determining the level of Selenoprotein P”, means that usually the immunoreactivity towards a region within the before mentioned molecules is determined. This means that it is not necessary that a certain fragment is measured selectively. It is understood that a binder which is used for the determination of the level of Selenoprotein P and/or fragments thereof binds to any fragment that comprises the region of binding of said binder. Said binder may be an antibody or antibody fragment or a non-IgG Scaffold.

In one specific embodiment the level of Selenoprotein P is measured with an immunoassay and said binder is an antibody, or an antibody fragment binding to Selenoprotein P and/or fragments thereof.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), chemiluminescence- and fluorescence-immunoassays, Luminex-based bead arrays, protein microarray assays, and rapid test formats such as for instance immunochromatographic strip tests (“dipstick immunoassays”) and immuno-chromatography assays.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

In one embodiment of the invention it may be a so-called POC-test (point-of-care) that is a test technology which allows performing the test within less than one hour near the patient without the requirement of a fully automated assay system. One example for this technology is the immunochromatographic test technology.

In one embodiment of the invention at least one of said two binders is labeled in order to be detected.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, auch as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′, 7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Chemiluminescent label may be acridinium ester label, steroid labels involving isoluminol labels and the like. Preferred chemiluminescent dyes are acridiniumesters.

Enzyme labels may be lactate dehydrogenase (LDH), creatine kinase (CPK), alkaline phosphatase, aspartate aminotransferase (AST), alanine aminotransferase (ALT), acid phosphatase, glucose-6-phosphate dehydrogenase and so on.

In one embodiment of the assays for determining Selenoprotein P and/or fragments thereof in a sample according to the present invention the assay sensitivity of said assay is <0.100 mg/L, preferably <0.05 mg/L and more preferably <0.01 mg/L.

According to the invention the diagnostic binder to Selenoprotein P and/or fragments thereof is selected from the group consisting of antibodies e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH₃ domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines.

In a specific embodiment the level of Selenoprotein P and/or fragments thereof are measured with an assay using binders selected from the group comprising an antibody, an antibody fragment, aptamers, non-Ig scaffolds as described in greater detail below binding to Selenoprotein P and/or fragments thereof.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is greater than 10⁷ M⁻¹, preferred 10⁸ M⁻¹, more preferred greater than 10⁹ M⁻¹, most preferred greater than 10¹⁰ M⁻¹. Binding affinity may be determined using the Biacore method, offered as service analysis e.g. at Biaffin, Kassel, Germany (http://www.biaffin.com/de/).

In the context of the present invention, “binder molecules” are molecules which may be used to bind target molecules or molecules of interest, i.e. analytes (i.e. in the context of the present invention Selenoprotein P and fragments thereof), from a sample. Binder molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

In addition to antibodies other biopolymer scaffolds are well known in the art to complex a target molecule and have been used for the generation of highly target specific biopolymers. Examples are aptamers, spiegelmers, anticalins and conotoxins. Non-Ig scaffolds may be protein scaffolds and may be used as antibody mimics as they are capable to bind to ligands or antigenes. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds (e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins preferably microproteins forming a cystine knot) scaffolds (e.g. described in EP 2314308), Fyn SH₃ domain based scaffolds (e.g. described in WO 2011/023685) EGFR-A-domain based scaffolds (e.g. described in WO 2005/040229) and Kunitz domain based scaffolds (e.g. described in EP 1941867).

In one embodiment of the invention at least one of said two binders is bound to a solid phase as magnetic particles, and polystyrene surfaces.

Alternatively, the level of any of the above analytes may be determined by other analytical methods e.g. mass spectroscopy.

In a specific embodiment of the methods of the present invention additionally at least one further biomarker is determined in the bodily fluid of a subject having heart failure and correlated with said (i) risk for getting a cardiovascular event and/or (ii) said risk of worsening heart failure condition and/or (iii) said risk for mortality and/or (iv) said risk of hospitalisation or re-hospitalisation due to heart failure as above outlined, wherein said additional biomarker is selected from the group comprising: pro-Neurotensin 1-117 (PNT 1-117), C-reactive protein (CRP), pro-brain natriuretic peptide 1-108 (proBNP 1-108, NT-proBNP), proBNP, BNP, pro-atrial natriuretic peptide 1-98 (proANP-N-terminal fragment), pro-ANP and fragments thereof of at least five amino acids in length (e.g. MR-proANP), adrenomedullin, pro-adrenomedullin (proADM) and fragments thereof of at least five amino acids in length (e.g. MR-proADM), ST-2, GDF15, Galectin-3, copeptin, human growth hormone (hGH), fasting blood or plasma glucose, triglycerides, HDL cholesterol or subfractions thereof, LDL cholesterol or subfractions thereof, insulin, cystatin C, selen, alanine-aminotranferase (ALT), aspartate-amino transferase (AST), bilirubin, alkaline phosphatase (ALP).

In a specific embodiment of the methods of the present invention additionally at least one further biomarker is determined in the bodily fluid of a subject having heart failure and correlated with said (i) risk for getting a cardiovascular event and/or (ii) said risk of worsening heart failure condition and/or (iii) said risk for mortality and/or (iv) said risk of hospitalisation or re-hospitalisation due to heart failure as above outlined, wherein said additional biomarker is selected from the group comprising: pro-Neurotensin 1-117 (PNT 1-117), C-reactive protein (CRP), pro-brain natriuretic peptide 1-108 (proBNP 1-108, NT-proBNP), proBNP, BNP, pro-atrial natriuretic peptide 1-98 (proANP-N-terminal fragment), pro-ANP and fragments thereof of at least five amino acids in length (e.g. MR-proANP), adrenomedullin, pro-adrenomedullin (proADM) and fragments thereof of at least five amino acids in length (e.g. MR-proADM), ST-2, GDF15, Galectin-3, copeptin, human growth hormone (hGH), fasting blood or plasma glucose, triglycerides, HDL cholesterol or subfractions thereof, LDL cholesterol or subfractions thereof, insulin, cystatin C, selen, alanine-aminotranferase (ALT), aspartate-amino transferase (AST), bilirubin, alkaline phosphatase (ALP), wherein the cardiovascular event is not stroke and wherein the cardiovascular mortality is not related to stroke.

Subject matter of the present invention is also a method for determining in a subject having heart failure the risk as defined in any of the preceding paragraphs: (i) the risk for getting a cardiovascular event and/or (ii) the risk of worsening heart failure condition and/or (iii) the risk for mortality and/or (iv) the risk hospitalisation or of re-hospitalisation due to having heart failure, wherein said method is performed in order to stratify said subjects into risk groups as further defined below.

Subject matter of the present invention is also a method for determining in a subject having heart failure the risk as defined in any of the preceding paragraphs: (i) the risk for getting a cardiovascular event and/or (ii) the risk of worsening heart failure condition and/or (iii) the risk for mortality and/or (iv) the risk hospitalisation or of re-hospitalisation due to having heart failure, wherein said method is performed in order to stratify said subjects into risk groups as further defined below, wherein the cardiovascular event is not stroke and wherein the cardiovascular mortality is not related to stroke.

In specific embodiments of the invention the methods are used in order to stratify the subjects into risk groups, e.g. those with a low risk, medium risk, or high. Low risk means that the value of Selenoprotein P and/or fragments thereof is substantially not decreased compared to a predetermined value in subjects who (i) did not get a cardiovascular event and/or (ii) did not get worsening heart failure condition and/or (iii) did not die within a certain period of time and/or (iv) did not get hospitalized or re-hospitalised due to heart failure. A medium risk exists when the level of Selenoprotein P and/or fragments thereof is elevated compared to a predetermined value in subjects who (i) did not get a cardiovascular event and/or (ii) did not get worsening heart failure condition and/or (iii) did not die within a certain period of time and/or (iv) did not get hospitalized or re-hospitalised due to heart failure, and a high risk exists when the level of Selenoprotein P and/or fragments thereof is significantly decreased at baseline measurement and continues to decrease at subsequent analysis compared to a predetermined value in subjects who did not get a cardiovascular event and/or (i) did not get worsening heart failure condition and/or (ii) did not die within a certain period of time and/or (iii) did not get hospitalized or re-hospitalised due to heart failure.

Fragments of Selenoprotein P may be selected from the group comprising SEQ ID No. 3 to 15.

A preventive therapy or intervention is the supplementation with selenium. Selenium may be applied as selenite, selenate or selenomethionine (L-selenomethionine).

The supplementation with selenium may be applied in combination with vitamins (e.g. vitamin E, vitamin C, vitamin A) and/or mineral nutrients (e.g. iodine, fluoride, zinc) and/or co-factors (e.g. coenzyme Q10).

Myocardial infarction, commonly known as a heart attack, occurs when blood flow decreases or stops to a part of the heart, causing damage to the heart muscle. The most common symptom is chest pain or discomfort, which may travel into the shoulder, arm, back, neck, or jaw. Myocardial infarction can be divided into ST-segment elevation myocardial infarction (STEMI) or non-ST-segment elevation myocardial infarction (NSTEMI).

Heart failure is a cardiac condition that occurs, when a problem with the structure or function of the heart impairs its ability to supply sufficient blood flow to meet the body's needs. It can cause a large variety of symptoms, particularly shortness of breath at rest or during exercise, signs of fluid retention such as pulmonary congestion or ankle swelling and objective evidence of an abnormality of the structure or function of the heart at rest. Acute heart failure is defined as a rapid onset of signs and symptoms of heart failure resulting in the need for urgent therapy or hospitalisation. Acute heart failure can present as acute de novo heart failure (new onset of acute heart failure in a patient without previous cardiac dysfunction) or acute decompensation of chronic heart failure.

Stroke is defined as an acute focal neurological deficit resulting from a cerebrovascular disease. The two main types of stroke are ischemic and hemorrhagic, accounting for approximately 85% and 15%, respectively.

As indicated above, in some specific embodiments the herein disclosed methods for assessing a risk in a subject having heart failure that is (i) the risk for getting a cardiovascular event and/or (ii) the risk of worsening heart failure condition and/or (iii) the risk for mortality, in particular cardiovascular mortality, and/or (iv) the risk of hospitalisation or re-hospitalisation due to heart failure, are not those, wherein stroke is the cardiovascular event or wherein the cardiovascular mortality is related to stroke.

Coronary re-vascularization includes percutaneous coronary intervention (PCI) and coronary artery bypass grafting (CABG). Percutaneous coronary intervention is a non-surgical procedure used to treat narrowing (stenosis) of the coronary arteries of the heart found in coronary artery disease. After accessing the blood stream through the femoral or radial artery, the procedure uses coronary catheterization to visualize the blood vessels on X-ray imaging. After this, an interventional cardiologist can perform a coronary angioplasty, using a balloon catheter in which a deflated balloon is advanced into the obstructed artery and inflated to relieve the narrowing; certain devices such as stents can be deployed to keep the blood vessel open. Various other procedures can also be performed. Coronary artery bypass surgery, also known as CABG surgery, and colloquially heart bypass or bypass surgery, is a surgical procedure to restore normal blood flow to an obstructed coronary artery. This surgery is often indicated when coronary arteries have a 50% to 99% obstruction.

Subject matter of the present invention is also the supplementation with selenium in subjects identified to be at high risk.

Solid dosage formulations for selenium are, e.g. tablets, capsules, granules, powders, sachets, reconstitutable powders, dry powder inhalers and chewables.

Further embodiments of the present invention:

With the above context, the following consecutively numbered embodiments provide further specific aspects of the invention:
1. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure, comprising
   a) determining the level and/or the amount of Selenoprotein P and/or fragments thereof in a sample of said subject,
   b) correlating the determined level and/or the amount of Selenoprotein P and/or fragments thereof in a subject having heart failure with (i) the risk for getting a cardiovascular event and/or (ii) with the risk of worsening heart failure condition and/or (iii) with the risk for mortality, and/or (iv) with the risk of hospitalisation or re-hospitalisation due to heart failure.
2. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalisation or re-hospitalisation due to heart failure according to item 1, wherein in a subject having heart failure (i) the risk for getting a cardiovascular event and/or (ii) the risk of worsening heart failure condition and/or (iii) the risk for mortality, and/or (iv) the risk of hospitalisation or re-hospitalisation due to heart failure is enhanced, when the determined level and/or the amount of Selenoprotein P and/or fragments thereof in a sample of said subject is below a threshold.
3. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalisation or re-hospitalisation in due to heart failure according to item 1 or 2, wherein in a subject having heart failure (i) the risk for getting a cardiovascular event and/or (ii) the risk of worsening heart failure condition and/or (iii) the risk for mortality, and/or (iv) the risk of hospitalisation or re-hospitalisation due to heart failure is enhanced when said and/or the amount of Selenoprotein P and/or fragments thereof in said sample is below a threshold, wherein said threshold is between 2.0 and 4.4 mg/L, preferably between 2.3 and 3.8 mg/L, more preferably between 2.6 and 3.4 mg/L, more preferably between 3.0 and 3.3 mg/L, most preferred said threshold is 3.3 mg/L.
4. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalisation or re-hospitalisation due to heart failure according to any of items 1-3, wherein in a subject having heart failure (i) the risk for getting a cardiovascular event and/or (ii) the risk of worsening heart failure condition and/or (iii) the risk for mortality, and/or (iv) the risk of hospitalisation or re-hospitalisation due to heart failure is enhanced when said level and/or the amount of Selenoprotein P and/or fragments thereof in said sample is below a threshold, wherein said threshold has been determined by the calculation of receiver operating characteristic curves (ROC curves), plotting the true positive rate (sensitivity, “disease” population e.g. subjects who did develop the condition) against the false positive rate (1-specificity, “normal” population e.g. subjects who did not develop the condition) at various threshold value settings.
5. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalisation or re-hospitalisation due to heart failure according to any of items 1-4, wherein in a subject having heart failure (i) the risk for getting a cardiovascular event and/or (ii) the risk of worsening heart failure condition and/or (iii) the risk for mortality, and/or (iv) the risk of hospitalisation or re-hospitalisation due to heart failure is enhanced when said level and/or the amount of Selenoprotein P and/or fragments thereof in said sample is below a threshold, wherein said threshold is the lower range of a heart failure population e.g. below 4.4 mg/L, more preferred below 3.8 mg/L, even more preferred below 3.4 mg/L, most preferred equal to or below 3.3 mg/L.
6. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalisation or re-hospitalisation due to heart failure according to any of items 1-5, wherein said cardiovascular event is selected from a group comprising myocardial infarction, stroke, coronary re-vascularization, and heart failure and said mortality is cardiovascular mortality.
7. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-6, wherein said mortality is cardiovascular mortality related to myocardial infarction, stroke or acute heart failure.
8. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-7, wherein said level and/or amount of Selenoprotein P and/or fragments thereof has been determined by an immunoassay using at least one binder binding to SEQ ID No. 2.
9. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to item 8, wherein said at least one binder is an antibody or a fragment thereof.
10. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-7, wherein said level and/or amount of Selenoprotein P and/or fragments thereof has been determined by mass spectroscopy.
11. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-10, wherein said (i) risk for getting a cardiovascular event and/or (ii) the risk of worsening heart failure condition and/or (iii) said risk for mortality is assessed for a period of time.
12. A method for assessing (i) the risk for getting a cardiovascular event and/or (ii) the risk of worsening heart failure condition and/or (iii) assessing the risk for mortality in a subject having heart failure and/or (iv) assessing the risk of re-hospitalisation in a hospitalized subject having heart failure according to any of items 1-11, wherein said (i) risk for getting a cardiovascular event and/or (ii) the risk of worsening heart failure condition and/or (iii) the risk for mortality in a subject having heart failure is assessed for a period of time of up to 1 year.
13. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-12, wherein said risk of hospitalisation or re-hospitalisation due to heart failure is assessed for a period of up to 30 days.
14. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-13, wherein the sample is a bodily fluid.
15. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalisation or re-hospitalisation due to heart failure according to any of items 1-14, wherein the sample is a bodily fluid selected from the group comprising whole blood, plasma, and serum.
16. Selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure.
17. Selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalisation or re-hospitalisation due to heart failure as determined according to a method of any of items 1-15.
18. Selenium for use in treatment of a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of re-hospitalisation due to heart failure as determined according to a method of any of items 1-15, wherein the determined level and/or the amount of Selenoprotein P and/or fragments thereof is below a threshold and wherein said threshold is between 2.0 and 4.4 mg/L.
19. In specific embodiments of the above items 1 to 18, the cardiovascular event is not stroke, and/or the cardiovascular mortality is not related to stroke.

### FIGURE DESCRIPTION

FIG. 1. Distribution of SePP (mg/L) within groups based on outcomes. A) no re-hospitalisation/survivor; B) re-hospitalised/survivor; C) no re-hospitalisation/deceased and D) re-hospitalised and deceased

FIG. 2. One-year survival within quartiles of SePP. Q1=quartile with lowest levels; Q4=quartile with highest levels. SePP levels within quartiles: Q1=1.8±0.4; Q2=2.6±0.2; Q3=3.4±0.2, Q4=4.7±0.8.

FIG. 3. 30-day re-hospitalisation within quartiles of SePP. Q1=quartile with lowest levels; Q4=quartile with highest levels. SePP levels within quartiles: Q1=1.8±0.4; Q2=2.6±0.2; Q3=3.4±0.2, Q4=4.7±0.8.

FIG. 4. Composite endpoint consisting of death or re-hospitalisation within 30 days, whichever came first, within quartiles of SePP. Q1=quartile with lowest levels; Q4=quartile with highest levels. SePP levels within quartiles: Q1=1.8±0.4; Q2=2.6±0.2; Q3=3.4±0.2, Q4=4.7±0.8.

FIG. 5. Comparison of the Selenoprotein P distribution in (A) a healthy reference population (MPP-study) and (B) heart failure patients (Harvest-study). Bold solid line shows the median of the respective population, broken line shows the first quartile of the healthy reference population.

### EXAMPLES

### Example 1: Assay Description

The Selenotest ELISA (Hybsier et al. 2017. Redox Biology 11: 403-414; Hybsier et al. 2015. Perspectives in Science 3: 23-24), a chromogenic enzyme-linked immunosorbent assay, for the quantitative determination of human Selenoproteinselenoprotein P in serum samples was used. The Selenotest ELISA is a sandwich enzyme immunoassay in 96 well plate format and uses two different Selenoproteinselenoprotein P specific monoclonal antibodies for the antigen capture and detection steps. The Selenoprotein P levels of the calibrators and controls were determined by measurements against serial dilutions of NIST SRM 1950 Standard Reference Material. Monoclonal antibodies (Ab) were generated by immunization of mice with an emulsion of purified recombinant Selenoprotein P. The specific monoclonal Ab5 was immobilized as capture-Ab, and the specific mAb2, was used as detection-Ab. The lower limit of quantification was determined at a Selenoprotein P level concentration of 11.6 μg/L, and the upper limit of quantification at 538.4 μg/L, thereby defining the working range at Selenoprotein P levels concentrations between 11.6 and 538.4 μg/L. The intersection at 20% CV defines the limit of detection, and was reached at a Selenoprotein P level concentration of 6.7 μg/L i.e., around 500-fold below average serum Selenoprotein P levels concentrations of well-supplied subjects. The signals were linear on dilution within the working range of the assay, and Selenoprotein P was stable in serum for 24 h at room temperature. For further details of the assay see Hybsier et al. 2017. Redox Biology 11: 403-414.

### Example 2: HARVEST-Malmö Study

The Swedish Heart and Brain Failure Investigation study (HARVEST-Malmo) is a prospective, on-going study undertaken in consecutive patients hospitalized for acute heart failure (either newly diagnosed or exacerbated chronic heart failure) in Malmö, Sweden. The only exclusion criterion was the inability to deliver consent. Baseline data including blood sample donations and clinical examination were collected between March 2014 and September 2018 in 324 subjects. Complete data was available in 295 patients. Data on one-year mortality (54 events), one-year cardiovascular-related mortality (44 events) and 30-day re-hospitalisation (61 events) was retrieved through national and regional registries. Selenoprotein P was measured upon admission, along with clinical examination.

### Clinical Examination

Upon hospitalisation, fasting blood samples were drawn, blood pressure was measured and body mass index (BMI) was calculated as kilograms per square meter. Subjects' health status (symptoms, function, and quality of life) was evaluated using Kansas City Cardiomyopathy Questionnaire (KCCQ), a valid and reliable measure of health status in both heart failure with reduced ejection fraction and heart failure with preserved ejection fraction (Joseph, Novak et al. 2013) an instrument that has been validated in Swedish (Patel, Ekman et al. 2008). Prevalent diabetes was defined as prior physician diagnosis of type 1 or type 2 diabetes, or use of antidiabetic medication. Atrial fibrillation (AF) was defined as prior diagnosis of HF. Prior congestive heart failure was defined as prior hospitalisation for congestive heart failure, or a physician diagnosis of heart failure prior to inclusion in the study.

### Laboratory Assays

Fasting N-terminal pro-brain natriuretic peptide (NT-proBNP) was analysed at the Department of Clinical Chemistry, Skåne University Hospital in Malmo, participating in a national standardisation and quality control system using a sandwich assay based on ElectroChemiLuminiscence Immunoassay (Cobas, Roche Diagnostic, Basel, Switzerland). As for analyses of Selenoprotein P, fasting blood samples were collected at admission in 4.5 ml EDTA-tubes and centrifuged at 1950 g for 10 minutes. Plasma was then aliquoted in 200 μl fractions into bar coded tubes (REMP, Brooks, Life Sciences, USA) and stored at −80° C. until analysis. Selenoprotein P was analysed with a validated ELISA immunoassay using monoclonal antibodies as described in Example 1.

### Outcomes

KCCQ was used to quantify physical limitations, symptoms, self-efficacy, social interference and quality of life. An overall summary score <50 was considered as an indication of low health-related quality of life, whereas overall summary scores ≥50 are an indication of better health-related quality of life (Soto, Jones et al. 2004). Total one-year mortality was defined as all-cause mortality within one year from study inclusion, and obtained from Swedish total population register Statistics Sweden. Re-hospitalisation was defined as first of any unplanned readmissions for worsening heart failure within 30 days from study inclusion. A composite endpoint of death or re-hospitalisation, whichever came first, within 30 days from study inclusion was created.

### Statistics

Prior to analyses, Selenoprotein P was normalized (z-standardised). NT-proBNP was the only variable with skewed distributions and therefore log transformed prior to analysis. Cross-sectional associations between Selenoprotein P and KCCQ were explored using logistic regression models, where the dependent variable KCCQ was dichotomized on <50 overall score points as a measure of low quality of life (higher points=better health-related quality of life) in a crude model, Model 1 (adjusted for age and sex) and Model 2 (further adjusted for BMI, systolic blood pressure (SBP), smoking, prevalent AF, prevalent diabetes, prior HF and log-transformed NT-proBNP). Cox regression models were carried out crude, in Model 1 (age and sex adjusted) and further adjusted for relevant risk factors in Model 2 (BMI, SBP, smoking, prevalent AF, prevalent diabetes, prior HF and log-transformed NT-proBNP) for one-year mortality, 30-day re-hospitalisation and a composite endpoint consisting of death or re-hospitalisation, whichever came first, within 30 days from study inclusion. Survival plots were computed using unadjusted Kaplan-Meier models. Length of hospital stay was analysed using crude linear regression models, adjusted for age and sex (Model 1) and further adjusted according to Model 2. All analyses were performed using IBM SPSS statistics version 25 (SPSS, Chicago, Ill.), except Harrell's C-statistics analyses that were performed using R 3.4.3.

A two-sided p-value <0.05 was considered statistically significant. Receiver operating curves (ROC) analysis was performed to determine threshold values with respective sensitivities and specificities.

### Results

Baseline characteristics of the study population within quartiles of Selenoprotein P levels are presented in Table 1. Selenoprotein P was normally distributed in the population (median 3.4 mg/L).

### Selenoprotein P and Quality of Life

Cross-sectional analyses of quality of life, based on a KCCQ overall score, revealed that each 1 SD increase of Selenoprotein P was associated with decreased risk of low health-related quality of life (n=47) defined as an overall summary score <50 in a crude model (OR 0.70; 95% CI 0.50-0.99, p=0.044), in Model 1 (OR 0.70; CI95% 0.50-0.99, p=0.043), and in fully adjusted Model 2 (OR 0.68; CI95% 0.47-0.97; p=0.035).

Due to disparities in sex between the lowest and the highest quartile of Selenoprotein P, an interaction analysis was performed. There was a significant interaction for sex, wherefore additional analyses were performed for each sex separately. Those revealed that the associations between Selenoprotein P levels and low health-related quality of life in the entire cohort were mainly driven by the male sex (n=205, 32 events, crude HR 0.67; CI 95% 0.45-0.99; p=0.048), whereas no significant associations were seen for females (n=89, 15 events, crude OR 0.82; CI 95% 0.40-1.67; p=0.581).

### Selenoprotein P and One-Year Mortality

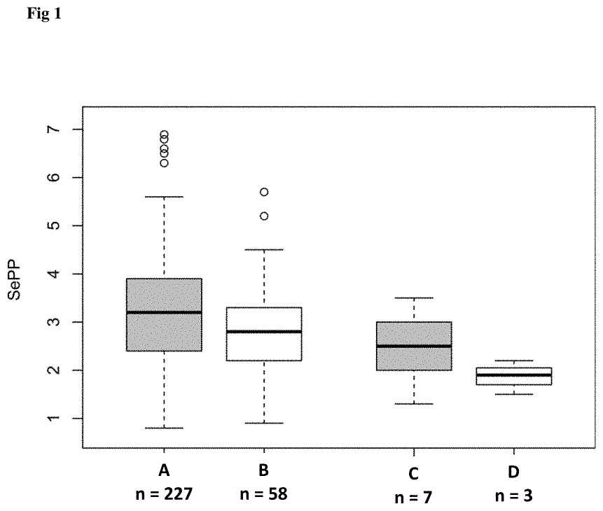

One-year mortality was higher in patients within the lowest quartile of Selenoprotein P (29.9%) as compared to patients with the highest levels of Selenoprotein P (8.8%). Selenoprotein P levels in relation to one-year mortality are illustrated in FIG. 1.

Cox regression analyses of one-year mortality are presented in Table 2, and reveal that each 1 SD increase in Selenoprotein P concentration was associated with lower risk of one-year mortality in crude analyses (HR 0.64; 95% CI 0.47-0.86; p=0.003), in Model 1 (HR 0.60; 95% CI 0.45-0.81, p=0.001), and further adjusted for BMI, SBP, smoking, prevalent AF, prevalent diabetes, log-transformed NT-proBNP and prior HF according to Model 2 (HR 0.65; 95% CI 0.48-0.88; p=0.005). C-index for Selenoprotein P was calculated to 0.628 (CI95% 0.553-0.703). Adding Selenoprotein P to the variables in Model 2 increases the (bootstrap-corrected) C-index from 0.736 to 0.751 (p for added value 0.004).

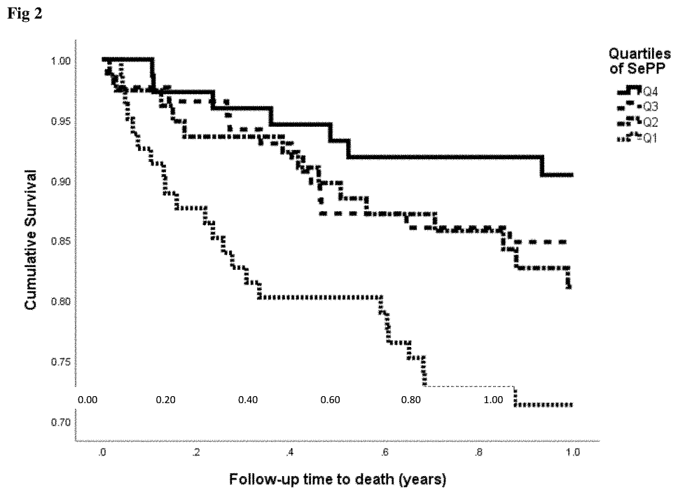

For better illustration, Selenoprotein P levels were divided into quartiles and related to one-year mortality (Table 3). Quartile analyses revealed that subjects in the quartile with lowest levels of Selenoprotein P (Q1) were at significantly higher risk of mortality within one year (HR 4.13; CI95% 1.64-10.4) as compared to subjects in Q4 (p for difference across quartiles 0.001) in fully adjusted Model 2. Kaplan Meier curves presenting survival within quartiles of Selenoprotein P are presented in FIG. 2.

Due to disparities in sex between the lowest and the highest quartile of Selenoprotein P, an interaction analysis was performed. There was a significant interaction for sex, wherefore additional analyses were performed for each sex separately. Those revealed that the associations between Selenoprotein P levels and mortality observed in the entire cohort were mainly driven by the male sex (n=208, 45 events, crude HR 0.60; CI 95% 0.44-0.82; p=0.001), whereas no significant associations were seen for females (n=92, 11 events, crude HR 0.72; CI 95% 0.35-1.52; p=0.391).

Exemplary threshold values for determining the risk of one-year mortality with respective sensitivities and specificities are shown in Table 4.

### Selenoprotein P and Risk of 30-Day Re-Hospitalisation

Thirty-day re-hospitalisation rate was higher in patients within the lowest quartile of Selenoprotein P (28.6%) as compared to patients with the highest levels of Selenoprotein P (7.4%). Selenoprotein P levels in relation to 30-day re-hospitalisation are illustrated in FIG. 1.

Cox regression analyses of 30-day re-hospitalisation (n=61) are presented in Table 2, and reveal that each 1 SD increment in Selenoprotein P concentration was associated with lower risk of re-hospitalisation within 30 days from study inclusion in crude analyses (HR 0.66; 95% CI 0.50-0.87; p=0.003), in Model 1 (HR 0.67; 95% CI 0.51-0.88, p=0.004), and further adjusted according to Model 2 (HR 0.67; 95% CI 0.51-0.89; p=0.005). C-index for Selenoprotein P was calculated to 0.617 (CI95% 0.552-0.682). Adding Selenoprotein P to the variables in Model 2 increases the C-ndex from 0.567 to 0.627 (p for added value 0.004). Bootstrap corrected C-index for model 2 is 0.48 (as none of the other variables contributes to prediction, the penalty is large and leaves the C-index below 0.5). Adding Selenoprotein P increases the boostrap corrected C-index to 0.547 (still less than Selenoprotein P alone, due to the penalty of adding 9 variables that have no predictive power).

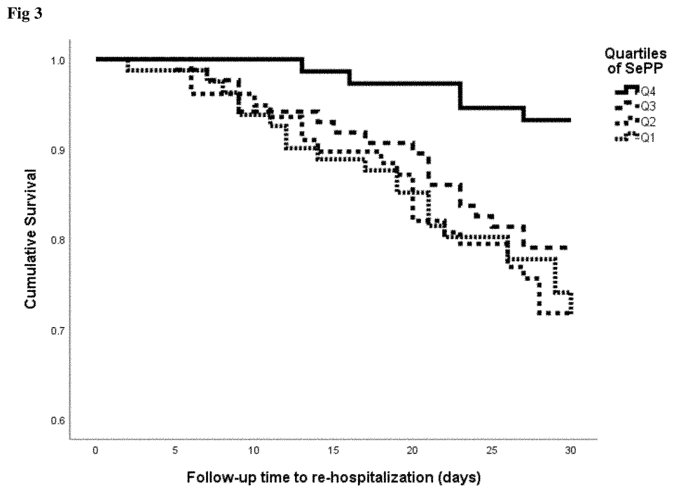

Additionally, Selenoprotein P levels were divided into quartiles and related to 30-day re-hospitalisation (Table 3). Quartile analyses revealed that subjects in the quartile with lowest levels of Selenoprotein P (Q1) were at significantly higher risk of re-hospitalisation within 30 days from study inclusion (HR 4.29; CI95% 1.59-11.6) as compared to subjects in Q4 (p for difference across quartiles 0.004) in fully adjusted Model 2. Kaplan Meier curves presenting re-hospitalisation within quartiles of Selenoprotein P are presented in FIG. 3.

Due to disparities in sex between the lowest and the highest quartile of Selenoprotein P, an interaction analysis was performed. There was a significant interaction for sex, wherefore additional analyses were performed for each sex separately. Those revealed that the associations between Selenoprotein P levels and 30-day re-hospitalisation observed in the entire cohort were mainly driven by the male sex (n=205, 39 events, crude HR 0.68; CI 95% 0.49-0.93; p=0.017), whereas no significant associations were seen for females (n=90, 22 events, crude HR 0.65; CI 95% 0.38-1.11; p=0.116).

Exemplary threshold values for determining the risk of 30-day re-hospitalisation with respective sensitivities and specificities are shown in Table 5.

Selenoprotein P and Composite Endpoint (Re-Hospitalisation or Death Within 30 Days)

Death or re-hospitalisation within 30 days from study inclusion was more frequent in patients within the lowest quartile of Selenoprotein P (32.4%) as compared to patients with the highest levels of Selenoprotein P (7.4%). Selenoprotein P levels in relation to the composite endpoint of death or re-hospitalisation are illustrated in FIG. 1.

Cox regression analyses of associations of Selenoprotein P and the composite endpoint (68 events) are presented in Table 2, and reveal that each 1 SD increase in Selenoprotein P concentration was associated with lower risk of either death or re-hospitalisation within 30 days in crude analyses (HR 0.64 CI95% 0.49-0.83, p=0.001), in Model 1 (HR 0.65; CI95% 0.50-0.85; p=0.001), and further adjusted for BMI, SBP, smoking, prevalent AF, prevalent diabetes, log-transformed NT-proBNP and prior HF according to Model 2 (HR 0.66; 0.51-0.86; p=0.002). C-index for Selenoprotein P was calculated to 0.622 (CI95% 0.562-0.681). Adding Selenoprotein P to the variables in Model 2 increases the C-index from 0.584 to 0.632 (p for added value 0.002). Bootstrap corrected C-index for Model 2 is 0.507 (as none of the variables contributes to prediction). Adding Selenoprotein P increases the boostrap corrected C-index to 0.561 (still less than Selenoprotein P alone, due to the penalty of adding 9 variables that have no predictive power).

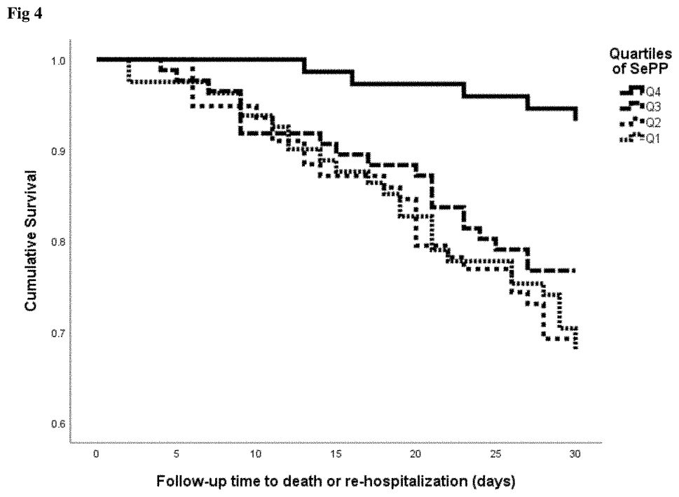

Further, Selenoprotein P levels were divided into quartiles and related to death or re-hospitalisation within 30 days (Table 3). Quartile analyses revealed that subjects in the quartile with lowest levels of Selenoprotein P (Q1) were at significantly higher risk of death or re-hospitalisation within 30 days (HR 4.80; CI95% 1.80-12.8) as compared to subjects in Q4 (p for difference across quartiles <0.002) in fully adjusted Model 2. Kaplan Meier curves presenting survival within quartiles of Selenoprotein P are presented in FIG. 4.

Distribution of Selenoprotein P within groups based on outcomes [A) no re-hospitalisation/survivor; B) re-hospitalised/survivor; C) no re-hospitalisation/deceased and D) re-hospitalised and deceased] is presented in FIG. 1.

### Hospital Stay

Further analyses were carried out for Selenoprotein P and length of hospital stay, where each 1 SD increase in Selenoprotein P levels was associated with shorter hospital stay in crude analyses (β−0.95, p<0.001), in Model 1 (β−1.04, p<0.001), and Model 2 (β−0.96, p<0.001).

### Discussion

This prospective study demonstrates that low plasma levels of Selenoprotein P are associated with lower health-related quality of life, higher one-year mortality risk, higher risk of 30-day readmission, and longer hospital stay upon admission for newly diagnosed or worsening acute heart failure. The prevalence of congestive heart failure, a common outcome to the majority of cardiac diseases, is steadily increasing worldwide, presumptively due to improved congestive heart failure survival and the ageing of the population (Savarese and Lund 2017).

The poor prognosis (Ponikowski, Voors et al. 2016), low quality of life (Hobbs, Kenkre et al. 2002) and the economic burden (Writing Group, Mozaffarian et al. 2016) posed to the society by the unplanned re-hospitalisations for worsening congestive heart failure make optimization of treatment of heart failure a top health priority. Up to date, no studies examining the associations of selenium deficiency (measured as low circulating levels of Selenoprotein P) and outcomes such as mortality and re-hospitalisation in a heart failure population have been presented.

Amongst 25 selenoproteins, Selenoprotein P has been suggested to act as a selenium transporter and to be essential in selenium metabolism and storage (Saito and Takahashi 2002, Labunskyy, Lee et al. 2011). In humans, levels of Selenoprotein P correlate with serum selenium levels (Andoh, Hirashima et al. 2005), and are usable as an index of the selenium nutritional status (Burk and Hill 2009). Selenium is an essential trace element that is involved in the control of the cell reduction-oxidation status and the immune system (McKenzie, Rafferty et al. 1998, Arthur, McKenzie et al. 2003, Huang, Rose et al. 2012), and is recognized as vital for the body's antioxidant defense mechanisms (Ahrens, Ellwanger et al. 2008). Increased oxidative stress has been proposed to contribute to pathogenesis of congestive heart failure (Givertz and Colucci 1998, Keith, Geranmayegan et al. 1998, Mallat, Philip et al. 1998, Singal, Khaper et al. 1998, Munzel and Harrison 1999, de Lorgeril and Salen 2006), and involvement of selenium in the protection from oxidative damage has been demonstrated in the cardiovascular system (Blankenberg, Rupprecht et al. 2003, Akbaraly, Arnaud et al. 2005, Ray, Semba et al. 2006, Joseph and Loscalzo 2013). As early as 1982, associations between low serum selenium levels and myocardial infarction and cardiovascular death were observed (Salonen, Alfthan et al. 1982). However, serum selenium is most likely a poor measure of the selenium status in the human body, whereas Selenoprotein P has been demonstrated to be valid biomarker of selenium status (Ashton, Hooper et al. 2009). In humans, Selenoprotein P has been demonstrated to be elevated in type 2 diabetes or prediabetes, as well as in overweight and obese subjects (Yang, Hwang et al. 2011) and Selenoprotein P expression levels have been shown to be severely up-regulated in subjects with type 2 diabetes (Misu, Takamura et al. 2010). No study has been able to conclude whether Selenoprotein P elevation in diabetes and prediabetes is a risk factor or a compensatory mechanism, given the fact that diabetes and insulin resistance are states of low-grade inflammation and oxidative stress. All of our analyses were adjusted for diabetes, implicating that associations of Selenoprotein P and low quality of life, one-year mortality and re-hospitalisation are independent of diabetes status.

In cardiovascular disease, selenium deficiency led to a larger myocardial injury after myocardial ischemia-reperfusion in rats (Venardos, Harrison et al. 2004), data in line with findings in other studies (Pucheu, Coudray et al. 1995, Toufektsian, Boucher et al. 2000, Tanguy, Toufektsian et al. 2003). Moreover, rats that received high selenium intake showed reduced infarct size, improved functional recovery of the heart and decreased incidence of ventricular arrhythmias (Tanguy, Boucher et al. 1998, Tanguy, Morel et al. 2004, Rakotovao, Tanguy et al. 2005, Tanguy, Rakotovao et al. 2011). Those findings might serve as plausible explanations to the findings in our study, due to the fact that the majority of all heart failure cases (>50% in the United States of America) (Gheorghiade and Bonow 1998)—primarily in males—are caused by underlying coronary artery disease. In our cohort, we lacked complete data on the subjects' heart failure etiology, and could thus not analyze the associations of selenium deficiency with different predisposing etiological causes.

In analyses of Selenoprotein P association with risk of one-year mortality, as well as 30-day re-hospitalisation and quality of life measured by KCCQ, an interaction with sex was observed. Sensitivity analyses were then performed for each sex separately, revealing that the associations observed were mainly driven by the male subjects. Nevertheless, those data need to be interpreted with great caution, in view of the lower event rate among females.

Although research has identified extensive selenium dependent functions in the human body, the role of selenium supplementation in cardiovascular disease remains uncertain (Flores-Mateo, Navas-Acien et al. 2006, Rees, Hartley et al. 2013). Up to date, there are no studies on effects of selenium supplementation on outcome in a population with acute heart failure, with Keshans disease as the only exception (McKeag, McKinley et al. 2012). Our findings urge for studies exploring effects of selenium supplementation on outcomes in heart failure.

There are both strengths and limitations to this study. As we consecutively included patients admitted for new or worsening heart failure with inability to deliver consent to the study as only exclusion criteria, we most probably mimicked a representative heart failure population.

All analyses were adjusted for clinically relevant risk factors, so we believe that the data demonstrate that selenium deficiency is prognostic of poor outcome in a congestive heart failure setting. Our data was collected at a single regional center, which limits the applicability to other population. Moreover, out sample size was relatively small and the results need to be replicated in larger cohorts. Also, the subjects included in HARVEST-Malmo were at mainly Swedish descent, and the conclusions drawn might not be generalizable to all ancestries.

### CONCLUSION

This study identifies Selenoprotein P as a novel marker of poor outcome in AHF and encourages future studies examining if supplementation of selenium might improve prognosis in CAHF-patients.

TABLE 1
Characteristics of the study population within quartiles of Selenoprotein P

| | Total | Q1 | Q2 | Q3 | Q4 | |
|---|---|---|---|---|---|---|
| | population | 2.0 (0.8-2.3) | 2.6 (2.3-3.0) | 3.4 (3.0-3.8) | 4.4 (3.8-6.9) | |
| | n = 295 | n = 77 | n = 71 | n = 79 | n = 68 | p |
| Age (years) | 74.4 ± 11.5 | 75.6 ± 10.5 | 73.8 ± 12.7 | 76.3 ± 10.9 | 73.0 ± 11.7 | 0.254 |
| Sex (female; n (%)) | 90 (30.5) | 29 (37.7) | 27 (38.0) | 22 (27.8) | 12 (17.6) | 0.025 |
| BMI (kg/m²) | 27.8 ± 6.0 | 27.6 ± 6.3 | 28.6 ± 7.2 | 27.0 ± 4.7 | 28.1 ± 5.5 | 0.425 |
| SBP (mmHg) | 136 ± 27 | 133 ± 23 | 138 ± 29 | 139 ± 25 | 136 ± 30 | 0.639 |
| Smoking (n (%)) | 37 (12.5) | 9 (11.7) | 10 (14.1) | 11 (13.9) | 7 (10.3) | 0.885 |
| Prevalent AF (n (%)) | 143 (48.5) | 37 (48.1) | 37 (52.1) | 41 (51.9) | 28 (41.2) | 0.535 |
| Prevalent diabetes (n (%)) | 109 (36.9) | 29 (37.7) | 22 (31.0) | 29 (36.7) | 29 (42.6) | 0.566 |
| Prior CHF (n (%)) | 194 (65.8) | 56 (72.7) | 47 (66.2) | 51 (64.6) | 40 (58.8) | 0.369 |
| SePP (mg/L) | 3.1 ± 1.1 | 1.8 ± 0.4 | 2.6 ± 0.2 | 3.4 ± 0.2 | 4.7 ± 0.7 | <0.001 |
| NT-proBNP pg/mL | 4096 (2234- | 4682 (2452- | 3768 (2378- | 4794 (2336- | 3118 (1795- | 0.181 |
| | 8645) | 11169) | 8862) | 7892) | 6200) | |
| KCCQ score (<50 points | 47 (15.9) | 13 (15.6) | 19 (26.8) | 5 (6.3) | 10 (14.7) | 0.008 |
| (%)) | | | | | | |
| One-year mortality (n (%)) | 54 (18.3) | 23 (29.9) | 12 (16.9) | 13 (16.5) | 6 (8.8) | 0.010 |
| Re-hospitalisation (n (%)) | 61 (20.7) | 22 (28.6) | 18 (25.4) | 16 (20.3) | 5 (7.4) | 0.010 |
| Composite endpoint | 68 (23.1) | 25 (32.5) | 20 (28.2) | 18 (22.7) | 5 (7.4) | 0.001 |
| (n (%)) | | | | | | |
| Hospital stay (days) | 7 (4-9) | 8 (5-10) | 7 (4-9) | 6 (4-8) | 6 (4-8) | 0.002 |

Values are means ± standard deviations (SD) or medians (interquartile range (25-75)) in the whole population and within quartiles of Selenoprotein P.
BMI = body mass index;
KCCQ = Kansas City Cardiomyopathy Questionnaire;
NT-proBNP = N-terminal prohormone of brain natriuretic peptide;
SBP = systolic blood pressure;
AF = atrial fibrillation;
CHF = congestive heart failure;
SePP = Selenoprotein P.
Q1 = quartile with lowest SePP levels;
Q4 = quartile with highest SePP levels.

TABLE 2
Associations of Selenoprotein P and risk of one-year mortality, 30-day
re-hospitalisation and major adverse outcomes

| | One-year mortality | | 30-day re-hospitalisation | | Composite endpoint | |
|---|---|---|---|---|---|---|
| | (n = 54) | | (n = 61) | | (n = 98) | |
| | HR (CI95%) | p | HR (CI95%) | p | HR (CI95%) | p |
| CRUDE | | | | | | |
| SePP | 0.64 (0.47-0.86) | 0.003 | 0.66 (0.50-0.87) | 0.003 | 0.65 (0.50-0.83) | 0.001 |
| MODEL 1 | | | | | | |
| Age | 1.06 (1.03-1.09) | <0.001 | 1.01 (0.98-1.03) | 0.540 | 1.01 (0.99-1.03) | 0.336 |
| Sex | 0.38 (0.19-0.74) | 0.004 | 1.13 (0.66-1.93) | 0.655 | 1.23 (0.75-2.04) | 0.523 |
| SePP | 0.60 (0.45-0.81) | 0.001 | 0.67 (0.51-0.88) | 0.004 | 0.65 (0.50-0.85) | 0.001 |
| MODEL 2 | | | | | | |
| Age | 1.07 (1.04-1.11) | <0.001 | 1.01 (0.98-1.04) | 0.524 | 1.02 (0.99-1.054 | 0.248 |
| Sex | 0.41 (0.21-0.82) | 0.012 | 1.14 (0.65-1.99) | 0.644 | 1.21 (0.72-2.05) | 0.471 |
| BMI | 0.99 (0.93-1.06) | 0.881 | 0.99 (0.94-1.05) | 0.779 | 1.01 (0.96-1.05) | 0.803 |
| SBP | 0.98 (0.97-0.99) | 0.001 | 1.00 (0.99-1.01) | 0.462 | 0.99 (0.98-1.00) | 0.217 |
| Smoking | 1.33 (0.51-3.50) | 0.562 | 1.03 (0.47-2.25) | 0.943 | 1.09 (0.52-2.23) | 0.820 |
| Prevalent AF | 0.59 (0.33-1.03) | 0.063 | 1.05 (0.63-1.76) | 0.852 | 0.99 (0.61-1.63) | 0.985 |
| Prevalent diabetes | 1.76 (0.96-3.23) | 0.068 | 0.97 (0.54-1.74) | 0.925 | 0.99 (0.57-1.72) | 0.204 |
| Prior CHF | 1.02 (0.51-2.06) | 0.948 | 1.25 (0.69-2.25) | 0.462 | 1.21 (0.69-2.11) | 0.697 |
| NT-proBNP | 1.46 (1.07-1.98) | 0.017 | 0.92 (0.70-1.19) | 0.516 | 0.96 (0.75-1.23) | 0.743 |
| SePP | 0.65 (0.48-0.88) | 0.005 | 0.67 (0.51-0.89) | 0.005 | 0.66 (0.51-0.86) | 0.002 |

Values are hazard ratios (HR) and 95% confidence intervals. BMI = body mass index; SBP = systolic blood pressure; AF = atrial fibrillation; CHF = congestive heart failure, SePP = Selenoprotein P. The composite endpoint is defined as death or re-hospitalisation within 30 days from study inclusion, whichever came first. Model 1 is adjusted for age and sex. Model 2 is adjusted for age, sex, body mass index, systolic blood pressure, log-transformed NT-proBNP, smoking, prevalent atrial fibrillation, prevalent diabetes and prior CHF.

TABLE 3
Quartile analyses of Selenoprotein P in relation to one-year mortality
and 30-day re-hospitalisation

| | One-year | 30-day re- | Composite |
|---|---|---|---|
| | mortality | hospitalisation | endpoint |
| | HR (CI95%) | HR (CI95%) | HR (CI95%) |
| Crude | | | |
| Q1 | 3.94 (1.60-9.67) | 4.35 (1.65-11.5) | 5.06 (1.94-13.2) |
| Q2 | 2.04 (0.76-5.42) | 3.90 (1.44-10.5) | 4.45 (1.67-11.9) |
| Q3 | 1.95 (0.74-5.12) | 2.97 (1.09-8.10) | 3.43 (1.27-9.24) |
| Q4 | Referent | Referent | Referent |
| p for trend | 0.001 | 0.002 | 0.001 |
| Model 1 | | | |
| Q1 | 4.66 (1.88-11.5) | 4.22 (1.59-11.2) | 4.80 (1.83-12.6) |
| Q2 | 2.26 (0.84-6.03) | 3.80 (1.40-10.3) | 4.26 (1.59-11.4) |
| Q3 | 1.77 (0.67-4.67) | 2.89 (1.05-7.89) | 3.29 (1.22-8.89) |
| Q4 | Referent | Referent | Referent |
| p for trend | <0.001 | 0.003 | 0.001 |
| Model 2 | | | |
| Q1 | 4.13 (1.64-10.43) | 4.29 (1.59-11.6) | 4.80 (1.80-12.8) |
| Q2 | 2.07 (0.76-5.63) | 3.87 (1.42-10.6) | 4.33 (1.60-11.7) |
| Q3 | 1.79 (0.67-4.81) | 2.97 (1.07-8.22) | 3.37 (1.24-9.20) |
| Q4 | Referent | Referent | Referent |
| p for trend | 0.001 | 0.004 | 0.002 |

Values are hazard ratios (HR) and 95% confidence intervals (95% CI) for quartiles of Selenoprotein P in relation to mortality within one year. Q1 = quartile with lowest levels of Selenoprotein P; Q4 = quartile with highest levels of Selenoprotein P. Model 1 is adjusted for age and sex. Model 2 is adjusted for age, sex, body mass index, systolic blood pressure, log-transformed NT-proBNP, smoking, prevalent atrial fibrillation, prevalent diabetes and prior CHF. Selenoprotein P levels within quartiles: Q1 (1.8 ± 0.4); Q2 (2.6 ± 0.2); Q3 (3.4 ± 0.2) Q4 (4.7 ± 0.8).

TABLE 4
Receiver operating curve (ROC) characteristics of Selenoprotein P
thresholds for one-year mortality with respective sensitivities
and specificities

| SePP (mg/L) | Specificity (in %) | Sensitivity (in %) |
|---|---|---|
| 2.0 | 87.6 | 27.8 |
| 2.3 | 77.6 | 42.6 |
| 2.6 | 62.7 | 50.0 |
| 3.0 | 53.1 | 64.8 |
| 3.3 | 43.2 | 79.6 |
| 3.8 | 25.7 | 88.9 |
| 4.4 | 14.5 | 90.7 |

TABLE 5
Receiver operating curve (ROC) characteristics of Selenoprotein P
thresholds for 30-days re-hospitalisation with respective
sensitivities and specificities

| SePP (mg/L) | Specificity (in %) | Sensitivity (in %) |
|---|---|---|
| 2.0 | 87.2 | 24.6 |
| 2.3 | 76.5 | 36.1 |
| 2.6 | 63.2 | 50.8 |
| 3.0 | 53.8 | 65.6 |
| 3.3 | 43.2 | 77.0 |
| 3.8 | 26.9 | 91.8 |
| 4.4 | 12.4 | 95.1 |

### Example 3: MPP-Study

### Study Description

The population-based Malmo Preventive Project (MPP) is a Swedish single-center prospective population-based study. Between 1974 and 1992, a total of 33,346 men and women of the homogenous ethnic background from the Malmo city area were recruited and screened for traditional risk factors of all-cause mortality and cardiovascular disease (CVD). A detailed description of baseline procedures may be found elsewhere (Fedorowski et al. 2010. Eur Heart J 31: 85-91; Berglund et al. 1996. J Intern Med 239: 489-97). In the years 2002-2006, all survivors from the original MPP cohort were invited for a reexamination. Of these, 18,240 participants (n=6,682 women) responded to the invitation and were reexamined including blood sampling and immediate −80° C. storage of EDTA plasma aliquots. The reexamination in 2002-2006 represents the baseline time point in the current study.

The 5060 of 18240 subjects tested for Selenoprotein P is a random sample (mean age 69 years). 4366 subjects were free from prior CVD (myocardial infarction, stroke and coronary re-vascularizations). Mean follow-up time of patients was 9.3 years, with deaths (n=1111), CVD deaths (n=351) and first CVD event (n=745). Selenoprotein P was measured with a validated ELISA immunoassay using monoclonal antibodies as described in Example 1. Baseline characteristics of the cohort are shown in table 6.

TABLE 6
Baseline characteristics of the MPP study population

| Variable | n = 4366 |
|---|---|
| Age | 69.4 (6.2) |
| gender male | 3008 (68.9%) |
| Current Smoking | 835 (19.1%) |
| AHT | 1476 (33.8%) |
| HDL | 1.4 (0.4) |
| LDL | 3.7 (1.0) |
| BMI | 27.1 (6.2) |
| SBP | 146.6 (20.3) |
| prevalent Diabetes | 466 (10.7%) |
| Deaths | 1111 (25.4%) |
| CVD Deaths | 351 (8%) |
| first CVD event | 745 (17.1%) |
| SePP (mg/L) | 5.5 (range 0.4-20.0) |

During a median (interquartile range) follow-up time of 9.3 (8.3-11) years, a total of 1111 deaths occurred. The largest number of deaths was observed in Selenoprotein P quintile 1 (n=314; 3.7 mg/L with a range between 0.4 and 4.3 mg/L). Similar patterns were observed for the endpoint analyses of cardiovascular mortality (351 events) and risk of a first cardiovascular event (745 events), respectively, with significantly higher risk in Selenoprotein P quintile 1.

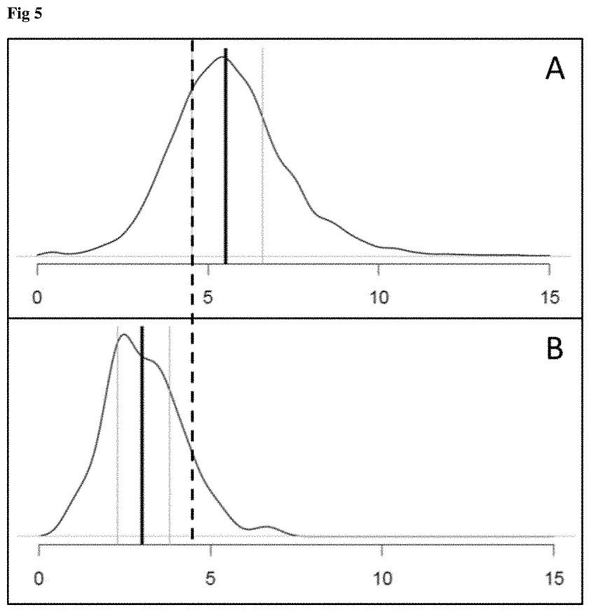

The frequency distribution of Selenoprotein P in this healthy population ranges from 0.4 to 20.0 mg/L with a median concentration of 5.5 mg/L (FIG. 5A). Threshold ranges of Selenoprotein P to assess the risk of healthy subjects for getting a first cardiovascular event or cardiovascular mortality are between 4.0 and 5.5 mg/L. When compared to the healthy population from MPP, the Selenoprotein P concentration of the heart failure population (HARVEST study), is a much lower concentration ranging between 0.8 and 6.9 mg/L and a median of 3.0 mg/L, where the majority of values are well below a threshold for healthy subjects (e.g. 97.3% of heart failure patients are below 5.5 mg/L and 79.7% of heart failure patients are below 4.0 mg/L) (FIG. 5B). Heart failure patients have Selenoprotein P concentrations that are compareable to healthy patients having a risk of getting a cardiovascular event, as those patients have already suffered a cardiovascular event (namely heart failure). Surprisingly, and according to the present invention the low Selenoprotein P concentrations in heart failure patients can further be divided into subgroups, whereas Selenoprotein P concentrations at the lower end of the distribution in heart failure patients have a higher risk of e.g. worsening heart failure or rehospitalization due to heart failure or mortality according to the present invention (see Example 2).

SEQUENCE LISTING

SEQ ID NO. 1: Selenoprotein P including signal sequence
(amino acid 1 to 381)
MWRSLGLALA LCLLPSGGTE SQDQSSLCKQ PPAWSIRDQD PMLNSNGSVT
VVALLQASUY LCILQASKLE DLRVKLKKEG YSNISYIVVN HQGISSRLKY
THLKNKVSEH IPVYQQEENQ TDVWTLLNGS KDDFLIYDRC GRLVYHLGLP
FSFLTFPYVE EAIKIAYCEK KCGNCSLTTL KDEDFCKRVS LATVDKTVET
PSPHYHHEHH HNHGHQHLGS SELSENQQPG APNAPTHPAP PGLHHHHKHK
GQHRQGHPEN RDMPASEDLQ DLQKKLCRKR CINQLLCKLP TDSELAPRSU
CCHCRHLIFE KTGSAITUQC KENLPSLCSU QGLRAEENIT ESCQURLPPA
AUQISQQLIP TEASASURUK NQAKKUEUPS N
SEQ ID NO. 2: secreted Selenoprotein P (amino acid 20 to 381)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCCHCRHLIF EKTGSAITUQ
CKENLPSLCS UQGLRAEENI TESCQURLPP AAUQISQQLI PTEASASURU
KNQAKKUEUP SN
SEQ ID NO. 3: Selenoprotein P (amino acid 20 to 346)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCCHCRHLIF EKTGSAITUQ
CKENLPSLCS UQGLRAEENI TESCQUR
SEQ ID NO. 4: Selenoprotein P (amino acid 20 to 298)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPR
SEQ ID NO. 5: Selenoprotein P (amino acid 20 to 299)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS
SEQ ID NO. 6: Selenoprotein P (amino acid 20 to 300)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS U
SEQ ID NO. 7: Selenoprotein P (amino acid 20 to 301)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UC
SEQ ID NO. 8: Selenoprotein P (amino acid 20 to 302)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCC
SEQ ID NO. 9: Selenoprotein P (amino acid 20 to 303)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCCH
SEQ ID NO. 10: Selenoprotein P (amino acid 20 to 304)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCCHC
SEQ ID NO. 11: Selenoprotein P (amino acid 20 to 305)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCCHCR
SEQ ID NO. 12: Selenoprotein P (amino acid 20 to 306)
ESQDQSSLCK QPPAWSIRDQ DPMLNSNGSV TVVALLQASU YLCILQASKL
EDLRVKLKKE GYSNISYIVV NHQGISSRLK YTHLKNKVSE HIPVYQQEEN
QTDVWTLLNG SKDDFLIYDR CGRLVYHLGL PFSFLTFPYV EEAIKIAYCE
KKCGNCSLTT LKDEDFCKRV SLATVDKTVE TPSPHYHHEH HHNHGHQHLG
SSELSENQQP GAPNAPTHPA PPGLHHHHKH KGQHRQGHPE NRDMPASEDL
QDLQKKLCRK RCINQLLCKL PTDSELAPRS UCCHCRH
SEQ ID NO. 13: Selenoprotein P (amino acid 1 to 235)
MWRSLGLALA LCLLPSGGTE SQDQSSLCKQ PPAWSIRDQD PMLNSNGSVT
VVALLQASUY LCILQASKLE DLRVKLKKEG YSNISYIVVN HQGISSRLKY
THLKNKVSEH IPVYQQEENQ TDVWTLLNGS KDDFLIYDRC GRLVYHLGLP
FSFLTFPYVE EAIKIAYCEK KCGNCSLTTL KDEDFCKRVS LATVDKTVET
PSPHYHHEHH HNHGHQHLGS SELSENQQPG APNAP
SEQ ID NO. 14: Selenoprotein P (amino acid 279 to 381)
KRCINQLLCK LPTDSELAPR SUCCHCRHLI FEKTGSAITU QCKENLPSLC
SUQGLRAEEN ITESCQURLP PAAUQISQQL IPTEASASUR UKNQAKKUEU PSN
SEQ ID NO. 15: Selenoprotein P (amino acid 312 to 381)
TGSAITUQCK ENLPSLCSUQ GLRAEENITE SCQURLPPAA UQISQQLIPT
EASASURUKN QAKKUEUPSN

### LITERATURE

Ahrens, I., C. Ellwanger, B. K. Smith, N. Bassler, Y. C. Chen, I. Neudorfer, A. Ludwig, C. Bode and K. Peter (2008). “Selenium supplementation induces metalloproteinase-dependent L-selectin shedding from monocytes.” J Leukoc Biol 83(6): 1388-1395.
Akbaraly, N. T., J. Arnaud, I. Hininger-Favier, V. Gourlet, A. M. Roussel and C. Berr (2005). “Selenium and mortality in the elderly: results from the EVA study.” Clin Chem 51(11): 2117-2123.
Akesson, B., T. Bellew and R. F. Burk (1994). “Purification of selenoprotein P from human plasma.” Biochim Biophys Acta 1204(2): 243-249.
Alehagen, U., J. Aaseth and P. Johansson (2015). “Reduced Cardiovascular Mortality 10 Years after Supplementation with Selenium and Coenzyme Q10 for Four Years: Follow-Up Results of a Prospective Randomized Double-Blind Placebo-Controlled Trial in Elderly Citizens.” PLoS One 10(12): e0141641.
Alehagen, U., P. Johansson, M. Bjornstedt, A. Rosen and U. Dahlstrom (2013). “Cardiovascular mortality and N-terminal-proBNP reduced after combined selenium and coenzyme Q10 supplementation: a 5-year prospective randomized double-blind placebo-controlled trial among elderly Swedish citizens.” Int J Cardiol 167(5): 1860-1866.
Andoh, A., M. Hirashima, H. Maeda, K. Hata, O. Inatomi, T. Tsujikawa, M. Sasaki, K. Takahashi and Y. Fujiyama (2005). “Serum selenoprotein-P levels in patients with inflammatory bowel disease.” Nutrition 21(5): 574-579.
Arroyo, M., S. P. Laguardia, S. K. Bhattacharya, M. D. Nelson, P. L. Johnson, L. D. Carbone, K. P. Newman and K. T. Weber (2006). “Micronutrients in African-Americans with decompensated and compensated heart failure.” Transl Res 148(6): 301-308.
Arthur, J. R., R. C. McKenzie and G. J. Beckett (2003). “Selenium in the immune system.” J Nutr 133(5 Suppl 1): 1457S-1459S.
Ashton, K., L. Hooper, L. J. Harvey, R. Hurst, A. Casgrain and S. J. Fairweather-Tait (2009). “Methods of assessment of selenium status in humans: a systematic review.” Am J Clin Nutr 89(6): 2025S-2039S.
Ballihaut, G., L. E. Kilpatrick, E. L. Kilpatrick and W. C. Davis (2012). “Multiple forms of selenoprotein P in a candidate human plasma standard reference material.” Metallomics 4(6): 533-538.
Blankenberg, S., H. J. Rupprecht, C. Bickel, M. Torzewski, G. Hafner, L. Tiret, M. Smieja, F. Cambien, J. Meyer, K. J. Lackner and I. AtheroGene (2003). “Glutathione peroxidase 1 activity and cardiovascular events in patients with coronary artery disease.” N Engl J Med 349(17): 1605-1613.
Bleys, J., A. Navas-Acien and E. Guallar (2008). “Serum selenium levels and all-cause, cancer, and cardiovascular mortality among US adults.” Arch Intern Med 168(4): 404-410.
Burk, R. F. and K. E. Hill (2009). “Selenoprotein P-expression, functions, and roles in mammals.” Biochim Biophys Acta 1790(11): 1441-1447.
Burk, R. F., B. K. Norsworthy, K. E. Hill, A. K. Motley and D. W. Byrne (2006). “Effects of chemical form of selenium on plasma biomarkers in a high-dose human supplementation trial.” Cancer Epidemiol Biomarkers Prev 15(4): 804-810.
Chen, M., B. Liu, D. Wilkinson, A. T. Hutchison, C. H. Thompson, G. A. Wittert and L. K. Heilbronn (2017). “Selenoprotein P is elevated in individuals with obesity, but is not independently associated with insulin resistance.” Obes Res Clin Pract 11(2): 227-232.
Clark, A. L., M. Cherif, T. A. McDonagh and I. B. Squire (2018). “In-hospital worsening heart failure: a clinically relevant endpoint?” ESC Heart Fail 5(1): 9-18.
de Lorgeril, M. and P. Salen (2006). “Selenium and antioxidant defenses as major mediators in the development of chronic heart failure.” Heart Fail Rev 11(1): 13-17.
Flores-Mateo, G., A. Navas-Acien, R. Pastor-Barriuso and E. Guallar (2006). “Selenium and coronary heart disease: a meta-analysis.” Am J Clin Nutr 84(4): 762-773.
Ghaemian, A., E. Salehifar, H. Shiraj and Z. Babaee (2012). “A Comparison of Selenium Concentrations between Congestive Heart Failure Patients and Healthy Volunteers.” J Tehran Heart Cent 7(2): 53-57.
Gharipour, M., M. Sadeghi, M. Salehi, M. Behmanesh, E. Khosravi, M. Dianatkhah, S. Haghjoo Javanmard, R. Razavi and A. Gharipour (2017). “Association of expression of selenoprotein P in mRNA and protein levels with metabolic syndrome in subjects with cardiovascular disease: Results of the Selenegene study.” J Gene Med 19(3).
Gheorghiade, M. and R. O. Bonow (1998). “Chronic heart failure in the United States: a manifestation of coronary artery disease.” Circulation 97(3): 282-289.
Givertz, M. M. and W. S. Colucci (1998). “New targets for heart-failure therapy: endothelin, inflammatory cytokines, and oxidative stress.” Lancet 352 Suppl 1: SI34-38.
Hill, K. E., R. S. Lloyd and R. F. Burk (1993). “Conserved nucleotide sequences in the open reading frame and 3′ untranslated region of selenoprotein P mRNA.” Proc Natl Acad Sci USA 90(2): 537-541.
Hill, K. E., Y. Xia, B. Akesson, M. E. Boeglin and R. F. Burk (1996). “Selenoprotein P concentration in plasma is an index of selenium status in selenium-deficient and selenium-supplemented Chinese subjects.” J Nutr 126(1): 138-145.
Hill, K. E., J. Zhou, L. M. Austin, A. K. Motley, A. J. Ham, G. E. Olson, J. F. Atkins, R. F. Gesteland and R. F. Burk (2007). “The selenium-rich C-terminal domain of mouse selenoprotein P is necessary for the supply of selenium to brain and testis but not for the maintenance of whole body selenium.” J Biol Chem 282(15): 10972-10980.
Himeno, S., H. S. Chittum and R. F. Burk (1996). “Isoforms of selenoprotein P in rat plasma. Evidence for a full-length form and another form that terminates at the second UGA in the open reading frame.” J Biol Chem 271(26): 15769-15775.
Hirashima, M., T. Naruse, H. Maeda, C. Nozaki, Y. Saito and K. Takahashi (2003). “Identification of selenoprotein P fragments as a cell-death inhibitory factor.” Biol Pharm Bull 26(6): 794-798.
Hobbs, F. D., J. E. Kenkre, A. K. Roalfe, R. C. Davis, R. Hare and M. K. Davies (2002). “Impact of heart failure and left ventricular systolic dysfunction on quality of life: a cross-sectional study comparing common chronic cardiac and medical disorders and a representative adult population.” Eur Heart J 23(23): 1867-1876.
Hollenbach, B., N. G. Morgenthaler, J. Struck, C. Alonso, A. Bergmann, J. Kohrle and L. Schomburg (2008). “New assay for the measurement of selenoprotein P as a sepsis biomarker from serum.” J Trace Elem Med Biol 22(1): 24-32.
Huang, Z., A. H. Rose and P. R. Hoffmann (2012). “The role of selenium in inflammation and immunity: from molecular mechanisms to therapeutic opportunities.” Antioxidants & redox signaling 16(7): 705-743.
Hybsier, S., T. Schulz, Z. Wu, I. Demuth, W. B. Minich, K. Renko, E. Rijntjes, J. Kohrle, C. J. Strasburger, E. Steinhagen-Thiessen and L. Schomburg (2017). “Sex-specific and inter-individual differences in biomarkers of selenium status identified by a calibrated ELISA for selenoprotein P.” Redox Biol 11: 403-414.
Joseph, J. and J. Loscalzo (2013). “Selenistasis: epistatic effects of selenium on cardiovascular phenotype.” Nutrients 5(2): 340-358.
Joseph, S. M., E. Novak, S. V. Arnold, P. G. Jones, H. Khattak, A. E. Platts, V. G. Davila-Roman, D. L. Mann and J. A. Spertus (2013). “Comparable performance of the Kansas City Cardiomyopathy Questionnaire in patients with heart failure with preserved and reduced ejection fraction.” Circ Heart Fail 6(6): 1139-1146.
Keith, M., A. Geranmayegan, M. J. Sole, R. Kurian, A. Robinson, A. S. Omran and K. N. Jeejeebhoy (1998). “Increased oxidative stress in patients with congestive heart failure.” J Am Coll Cardiol 31(6): 1352-1356.
Kosar, F., I. Sahin, C. Taskapan, Z. Kucukbay, H. Gullu, H. Taskapan and S. Cehreli (2006). “Trace element status (Se, Zn, Cu) in heart failure.” Anadolu Kardiyol Derg 6(3): 216-220.
Labunskyy, V. M., B. C. Lee, D. E. Handy, J. Loscalzo, D. L. Hatfield and V. N. Gladyshev (2011). “Both maximal expression of selenoproteins and selenoprotein deficiency can promote development of type 2 diabetes-like phenotype in mice.” Antioxid Redox Signal 14(12): 2327-2336.
Liu, H., H. Xu and K. Huang (2017). “Selenium in the prevention of atherosclerosis and its underlying mechanisms.” Metallomics 9(1): 21-37.
Lubos, E., C. R. Sinning, R. B. Schnabel, P. S. Wild, T. Zeller, H. J. Rupprecht, C. Bickel, K. J. Lackner, D. Peetz, J. Loscalzo, T. Munzel and S. Blankenberg (2010). “Serum selenium and prognosis in cardiovascular disease: results from the AtheroGene study.” Atherosclerosis 209(1): 271-277.
Ma, S., K. E. Hill, R. M. Caprioli and R. F. Burk (2002). “Mass spectrometric characterization of full-length rat selenoprotein P and three isoforms shortened at the C terminus. Evidence that three UGA codons in the mRNA open reading frame have alternative functions of specifying selenocysteine insertion or translation termination.” J Biol Chem 277(15): 12749-12754.
Mallat, Z., I. Philip, M. Lebret, D. Chatel, J. Maclouf and A. Tedgui (1998). “Elevated levels of 8-iso-prostaglandin F2alpha in pericardial fluid of patients with heart failure: a potential role for in vivo oxidant stress in ventricular dilatation and progression to heart failure.” Circulation 97(16): 1536-1539.
McKeag, N. A., M. C. McKinley, J. V. Woodside, M. T. Harbinson and P. P. McKeown (2012). “The role of micronutrients in heart failure.” J Acad Nutr Diet 112(6): 870-886.
McKenzie, R. C., T. S. Rafferty and G. J. Beckett (1998). “Selenium: an essential element for immune function.” Immunol Today 19(8): 342-345.
Meplan, C., L. K. Crosley, F. Nicol, G. J. Beckett, A. F. Howie, K. E. Hill, G. Horgan, J. C. Mathers, J. R. Arthur and J. E. Hesketh (2007). “Genetic polymorphisms in the human selenoprotein P gene determine the response of selenoprotein markers to selenium supplementation in a gender-specific manner (the SELGEN study).” Faseb j 21(12): 3063-3074.
Misu, H., T. Takamura, H. Takayama, H. Hayashi, N. Matsuzawa-Nagata, S. Kurita, K. Ishikura, H. Ando, Y. Takeshita, T. Ota, M. Sakurai, T. Yamashita, E. Mizukoshi, T. Yamashita, M. Honda, K. Miyamoto, T. Kubota, N. Kubota, T. Kadowaki, H. J. Kim, I. K. Lee, Y. Minokoshi, Y. Saito, K. Takahashi, Y. Yamada, N. Takakura and S. Kaneko (2010). “A liver-derived secretory protein, selenoprotein P, causes insulin resistance.” Cell Metab 12(5): 483-495.
Munzel, T. and D. G. Harrison (1999). “Increased superoxide in heart failure: a biochemical baroreflex gone awry.” Circulation 100(3): 216-218.
Patel, H., I. Ekman, J. A. Spertus, S. M. Wasserman and L. O. Persson (2008). “Psychometric properties of a Swedish version of the Kansas City Cardiomyopathy Questionnaire in a Chronic Heart Failure population.” Eur J Cardiovasc Nurs 7(3): 214-221.
Ponikowski, P., A. A. Voors, S. D. Anker, H. Bueno, J. G. F. Cleland, A. J. S. Coats, V. Falk, J. R. Gonzalez-Juanatey, V. P. Harjola, E. A. Jankowska, M. Jessup, C. Linde, P. Nihoyannopoulos, J. T. Parissis, B. Pieske, J. P. Riley, G. M. C. Rosano, L. M. Ruilope, F. Ruschitzka, F. H. Rutten, P. van der Meer and E. S. C. S. D. Group (2016). “2016 ESC Guidelines for the diagnosis and treatment of acute and chronic heart failure: The Task Force for the diagnosis and treatment of acute and chronic heart failure of the European Society of Cardiology (ESC) Developed with the special contribution of the Heart Failure Association (HFA) of the ESC.” Eur Heart J 37(27): 2129-2200.
Pucheu, S., C. Coudray, N. Tresallet, A. Favier and J. de Leiris (1995). “Effect of dietary antioxidant trace element supply on cardiac tolerance to ischemia-reperfusion in the rat.” J Mol Cell Cardiol 27(10): 2303-2314.
Rakotovao, A., S. Tanguy, M. C. Toufektsian, C. Berthonneche, V. Ducros, A. Tosaki, J. de Leiris and F. Boucher (2005). “Selenium status as determinant of connexin-43 dephosphorylation in ex vivo ischemic/reperfused rat myocardium.” J Trace Elem Med Biol 19(1): 43-47.
Ray, A. L., R. D. Semba, J. Walston, L. Ferrucci, A. R. Cappola, M. O. Ricks, Q. L. Xue and L. P. Fried (2006). “Low serum selenium and total carotenoids predict mortality among older women living in the community: the women's health and aging studies.” J Nutr 136(1): 172-176.
Rayman, M. P. and S. Stranges (2013). “Epidemiology of selenium and type 2 diabetes: can we make sense of it?” Free Radic Biol Med 65: 1557-1564.
Read, R., T. Bellew, J. G. Yang, K. E. Hill, I. S. Palmer and R. F. Burk (1990). “Selenium and amino acid composition of selenoprotein P, the major selenoprotein in rat serum.” J Biol Chem 265(29): 17899-17905.
Rees, K., L. Hartley, C. Day, N. Flowers, A. Clarke and S. Stranges (2013). “Selenium supplementation for the primary prevention of cardiovascular disease.” Cochrane Database Syst Rev(1): Cd009671.
Reeves, M. A. and P. R. Hoffmann (2009). “The human selenoproteome: recent insights into functions and regulation.” Cell Mol Life Sci 66(15): 2457-2478.
Renko, K., P. J. Hofmann, M. Stoedter, B. Hollenbach, T. Behrends, J. Kohrle, U. Schweizer and L. Schomburg (2009). “Down-regulation of the hepatic selenoprotein biosynthesis machinery impairs selenium metabolism during the acute phase response in mice.” Faseb j 23(6): 1758-1765.
Renko, K., M. Werner, I. Renner-Muller, T. G. Cooper, C. H. Yeung, B. Hollenbach, M. Scharpf, J. Kohrle, L. Schomburg and U. Schweizer (2008). “Hepatic selenoprotein P (SePP) expression restores selenium transport and prevents infertility and motor-incoordination in Sepp-knockout mice.” Biochem J 409(3): 741-749.
Saito, Y. and K. Takahashi (2002). “Characterization of selenoprotein P as a selenium supply protein.” Eur J Biochem 269(22): 5746-5751.
Saliba, W., R. El Fakih and W. Shaheen (2010). “Heart failure secondary to selenium deficiency, reversible after supplementation.” Int J Cardiol 141(2): e26-27.
Salonen, J. T., G. Alfthan, J. K. Huttunen, J. Pikkarainen and P. Puska (1982). “Association between cardiovascular death and myocardial infarction and serum selenium in a matched-pair longitudinal study.” Lancet 2(8291): 175-179.
Savarese, G. and L. H. Lund (2017). “Global Public Health Burden of Heart Failure.” Cardiac failure review 3(1): 7-11.
Singal, P. K., N. Khaper, V. Palace and D. Kumar (1998). “The role of oxidative stress in the genesis of heart disease.” Cardiovasc Res 40(3): 426-432.
Soto, G. E., P. Jones, W. S. Weintraub, H. M. Krumholz and J. A. Spertus (2004). “Prognostic value of health status in patients with heart failure after acute myocardial infarction.” Circulation 110(5): 546-551.
Strauss, E., J. Tomczak, R. Staniszewski and G. Oszkinis (2018). “Associations and interactions between variants in selenoprotein genes, selenoprotein levels and the development of abdominal aortic aneurysm, peripheral arterial disease, and heart failure.” PLoS One 13(9): e0203350.
Tanguy, S., F. Boucher, S. Besse, V. Ducros, A. Favier and J. de Leiris (1998). “Trace elements and cardioprotection: increasing endogenous glutathione peroxidase activity by oral selenium supplementation in rats limits reperfusion-induced arrhythmias.” J Trace Elem Med Biol 12(1): 28-38.
Tanguy, S., S. Morel, C. Berthonneche, M. C. Toufektsian, M. de Lorgeril, V. Ducros, A. Tosaki, J. de Leiris and F. Boucher (2004). “Preischemic selenium status as a major determinant of myocardial infarct size in vivo in rats.” Antioxid Redox Signal 6(4): 792-796.
Tanguy, S., A. Rakotovao, M. G. Jouan, C. Ghezzi, J. de Leiris and F. Boucher (2011). “Dietary selenium intake influences Cx43 dephosphorylation, TNF-alpha expression and cardiac remodeling after reperfused infarction.” Mol Nutr Food Res 55(4): 522-529.
Tanguy, S., M. C. Toufektsian, S. Besse, V. Ducros, J. De Leiris and F. Boucher (2003). “Dietary selenium intake affects cardiac susceptibility to ischaemia/reperfusion in male senescent rats.” Age Ageing 32(3): 273-278.
Toufektsian, M. C., F. Boucher, S. Pucheu, S. Tanguy, C. Ribuot, D. Sanou, N. Tresallet and J. de Leiris (2000). “Effects of selenium deficiency on the response of cardiac tissue to ischemia and reperfusion.” Toxicology 148(2-3): 125-132.
Venardos, K., G. Harrison, J. Headrick and A. Perkins (2004). “Effects of dietary selenium on glutathione peroxidase and thioredoxin reductase activity and recovery from cardiac ischemia-reperfusion.” J Trace Elem Med Biol 18(1): 81-88.
Writing Group, M., D. Mozaffarian, E. J. Benjamin, A. S. Go, D. K. Arnett, M. J. Blaha, M. Cushman, S. R. Das, S. de Ferranti, J. P. Despres, H. J. Fullerton, V. J. Howard, M. D. Huffman, C. R. Isasi, M. C. Jimenez, S. E. Judd, B. M. Kissela, J. H. Lichtman, L. D. Lisabeth, S. Liu, R. H. Mackey, D. J. Magid, D. K. McGuire, E. R. Mohler, 3rd, C. S. Moy, P. Muntner, M. E. Mussolino, K. Nasir, R. W. Neumar, G. Nichol, L. Palaniappan, D. K. Pandey, M. J. Reeves, C. J. Rodriguez, W. Rosamond, P. D. Sorlie, J. Stein, A. Towfighi, T. N. Turan, S. S. Virani, D. Woo, R. W. Yeh, M. B. Turner, C. American Heart Association Statistics and S. Stroke Statistics (2016). “Heart Disease and Stroke Statistics-2016 Update: A Report From the American Heart Association.” Circulation 133(4): e38-360.
Xia, Y., K. E. Hill, D. W. Byrne, J. Xu and R. F. Burk (2005). “Effectiveness of selenium supplements in a low-selenium area of China.” Am J Clin Nutr 81(4): 829-834.
Yang, J. G., K. E. Hill and R. F. Burk (1989). “Dietary selenium intake controls rat plasma selenoprotein P concentration.” J Nutr 119(7): 1010-1012.
Yang, S. J., S. Y. Hwang, H. Y. Choi, H. J. Yoo, J. A. Seo, S. G. Kim, N. H. Kim, S. H. Baik, D. S. Choi and K. M. Choi (2011). “Serum selenoprotein P levels in patients with type 2 diabetes and prediabetes: implications for insulin resistance, inflammation, and atherosclerosis.” J Clin Endocrinol Metab 96(8): E1325-1329.
Yang, X., K. E. Hill, M. J. Maguire and R. F. Burk (2000). “Synthesis and secretion of selenoprotein P by cultured rat astrocytes.” Biochim Biophys Acta 1474(3): 390-396.

1. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure, comprising
a) determining the level and/or the amount of Selenoprotein P and/or fragments thereof in a sample of said subject,
b) correlating the determined level and/or the amount of Selenoprotein P and/or fragments thereof in a subject having heart failure with (i) the risk for getting a cardiovascular event and/or (ii) with the risk of worsening heart failure condition and/or (iii) with the risk for mortality, and/or (iv) with the risk of hospitalization or re-hospitalization due to heart failure.

2. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalization or re-hospitalization due to heart failure according to claim 1, wherein in a subject having heart failure (i) the risk for getting a cardiovascular event and/or (ii) the risk of worsening heart failure condition and/or (iii) the risk for mortality, and/or (iv) the risk of hospitalization or re-hospitalization due to heart failure is enhanced, when the determined level and/or the amount of Selenoprotein P and/or fragments thereof in a sample of said subject is below a threshold.

3. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalization or re-hospitalization in due to heart failure according to claim 1, wherein in a subject having heart failure (i) the risk for getting a cardiovascular event and/or (ii) the risk of worsening heart failure condition and/or (iii) the risk for mortality, and/or (iv) the risk of hospitalization or re-hospitalization due to heart failure is enhanced when said and/or the amount of Selenoprotein P and/or fragments thereof in said sample is below a threshold, wherein said threshold is between 2.0 and 4.4 mg/L, preferably between 2.3 and 3.8 mg/L, more preferably between 2.6 and 3.4 mg/L, more preferably between 3.0 and 3.3 mg/L, most preferred said threshold is 3.3 mg/L.

4. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalization or re-hospitalization due to heart failure according to claim 1, wherein in a subject having heart failure (i) the risk for getting a cardiovascular event and/or (ii) the risk of worsening heart failure condition and/or (iii) the risk for mortality, and/or (iv) the risk of hospitalization or re-hospitalization due to heart failure is enhanced when said level and/or the amount of Selenoprotein P and/or fragments thereof in said sample is below a threshold, wherein said threshold has been determined by the calculation of receiver operating characteristic curves (ROC curves), plotting the true positive rate (sensitivity, “disease” population e.g. subjects who did develop the condition) against the false positive rate (1-specificity, “normal” population e.g. subjects who did not develop the condition) at various threshold value settings.

5. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalization or re-hospitalization due to heart failure according to claim 1, wherein in a subject having heart failure (i) the risk for getting a cardiovascular event and/or (ii) the risk of worsening heart failure condition and/or (iii) the risk for mortality, and/or (iv) the risk of hospitalization or re-hospitalization due to heart failure is enhanced when said level and/or the amount of Selenoprotein P and/or fragments thereof in said sample is below a threshold, wherein said threshold is the lower range of a heart failure population e.g. below 4.4 mg/L, more preferred below 3.8 mg/L, even more preferred below 3.4 mg/L, most preferred equal to or below 3.3 mg/L.

6. A method for assessing a risk in a subject having heart failure that is (i) the risk for getting a cardiovascular event and/or (ii) the risk of worsening heart failure condition and/or (iii) assessing the risk for mortality, and/or (iv) assessing the risk of hospitalization or re-hospitalization due to heart failure according to claim 1, wherein said cardiovascular event is selected from a group comprising myocardial infarction, stroke, coronary re-vascularization, and heart failure and said mortality is cardiovascular mortality.

7. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein said mortality is cardiovascular mortality related to myocardial infarction, stroke or acute heart failure.

8. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein said level and/or amount of Selenoprotein P and/or fragments thereof has been determined by an immunoassay using at least one binder binding to SEQ ID No. 2.

9. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 8, wherein said at least one binder is an antibody or a fragment thereof.

10. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein said level and/or amount of Selenoprotein P and/or fragments thereof has been determined by mass spectroscopy.

11. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein said (i) risk for getting a cardiovascular event and/or (ii) the risk of worsening heart failure condition and/or (iii) said risk for mortality is assessed for a period of time of up to one year.

12. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein said risk of hospitalization or re-hospitalization due to heart failure is assessed for a period of up to 30 days.

13. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein the sample is a bodily fluid.

14. A method for assessing a risk in a subject having heart failure that is (i) the risk for getting a cardiovascular event and/or (ii) the risk of worsening heart failure condition and/or (iii) assessing the risk for mortality and/or (iv) assessing the risk of hospitalization or re-hospitalization due to heart failure according to claim 1, wherein the sample is a bodily fluid selected from the group comprising whole blood, plasma, and serum.

15. A method for treating a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of hospitalization or re-hospitalization due to heart failure, comprising administering selenium to said subject.

16. A method of treating a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk hospitalization or re-hospitalization due to heart failure as determined according to a method of claim 1, comprising administering selenium to said subject.

17. A method of treating a subject having heart failure and having an enhanced risk for (i) getting a cardiovascular event and/or (ii) having an enhanced risk for worsening heart failure condition and/or (iii) having an enhanced risk for mortality and/or (iv) having an enhanced risk of re-hospitalization due to heart failure as determined according to a method of claim 1, comprising: administering selenium to said subject, and wherein the determined level and/or the amount of Selenoprotein P and/or fragments thereof is below a threshold and wherein said threshold is between 2.0 and 4.4 mg/L.


SEQUENCE LISTING (15 sequences)

SEQ ID NO: 1 - protein, 381 aa, Homo sapiens
  UNSURE (1)..(381): Xaa or single letter X represents Selenocystein (U) in originally disclosed sequence
MWRSLGLALALCLLPSGGTESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCRHLIFEKTGSAITXQCKENLPSLCSXQGLRAEENITESCQXRLPPAAXQISQQLIPTEASASXRXKNQAKKXEXPSN

SEQ ID NO: 2 - protein, 362 aa, Homo sapiens
  UNSURE (1)..(362): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCRHLIFEKTGSAITXQCKENLPSLCSXQGLRAEENITESCQXRLPPAAXQISQQLIPTEASASXRXKNQAKKXEXPSN

SEQ ID NO: 3 - protein, 327 aa, Homo sapiens
  UNSURE (1)..(327): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCRHLIFEKTGSAITXQCKENLPSLCSXQGLRAEENITESCQXR

SEQ ID NO: 4 - protein, 279 aa, Homo sapiens
  UNSURE (1)..(279): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPR

SEQ ID NO: 5 - protein, 280 aa, Homo sapiens
  UNSURE (1)..(280): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRS

SEQ ID NO: 6 - protein, 281 aa, Homo sapiens
  UNSURE (1)..(281): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSX

SEQ ID NO: 7 - protein, 282 aa, Homo sapiens
  UNSURE (1)..(282): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXC

SEQ ID NO: 8 - protein, 283 aa, Homo sapiens
  UNSURE (1)..(283): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCC

SEQ ID NO: 9 - protein, 284 aa, Homo sapiens
  UNSURE (1)..(284): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCH

SEQ ID NO: 10 - protein, 285 aa, Homo sapiens
  UNSURE (1)..(285): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHC

SEQ ID NO: 11 - protein, 286 aa, Homo sapiens
  UNSURE (1)..(286): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCR

SEQ ID NO: 12 - protein, 287 aa, Homo sapiens
  UNSURE (1)..(287): Xaa or single letter X represents Selenocystein (U) in originally disclosed sequence
ESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCRH

SEQ ID NO: 13 - protein, 235 aa, Homo sapiens
  UNSURE (1)..(235): Xaa or single letter X represents Selenocystein (U) in originally disclosed sequence
MWRSLGLALALCLLPSGGTESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAP

SEQ ID NO: 14 - protein, 103 aa, Homo sapiens
  UNSURE (1)..(103): Xaa or single letter X represents Selenocystein (U) in originally disclosed sequence
KRCINQLLCKLPTDSELAPRSXCCHCRHLIFEKTGSAITXQCKENLPSLCSXQGLRAEENITESCQXRLPPAAXQISQQLIPTEASASXRXKNQAKKXEXPSN

SEQ ID NO: 15 - protein, 70 aa, Homo sapiens
  UNSURE (1)..(70): Xaa or single letter X represents Selenocystein (U) in originally disclosed sequence
TGSAITXQCKENLPSLCSXQGLRAEENITESCQXRLPPAAXQISQQLIPTEASASXRXKNQAKKXEXPSN